(19) United States
(12) Patent Application Publication
(10) Pub. No.: US 2026/0325469 A1
(43) Pub. Date: Oct. 22, 2026

(54) COMPOSITION FOR ANIMALS

(71) Applicant: NERAN APS, Fredensborg (DK)

(72) Inventor: Ahrnt CLAUSEN, Fredensborg (DK)

(21) Appl. No.: 19/476,594

(22) PCT Filed: Apr. 19, 2024
(86) PCT No.: PCT/DK2024/050094

§ 371 Date: Oct. 17, 2025

(30) Foreign Application Priority Data
Apr. 21, 2023 (DK) PA 2023 70190

(51) Int. Cl. A61K 31/19; A61K 9/08; A61K 31/7024; A61K 31/731; A61P 1/12; A61P 3/00; A61P 31/04

(52) U.S. Cl. CPC A61K 31/19; A61K 9/08; A61K 31/7024; A61K 31/731; A61P 1/12; A61P 3/00; A61P 31/04

(57) ABSTRACT

The present invention relates to a composition for animals comprising a combination of one, two, or more acid component(s) and a binding agent. The binding agent comprise a tannin component and a polygalactose component. Further, the present invention relates to a composition for use as an alternative to medicine and, in particular, for use in preventing and/or treating diarrhoea in an animal. The present invention also relates to the use of the composition for reducing the use of antibiotics and/or zinc oxide in animal farming industry.

### TECHNICAL FIELD OF THE INVENTION

The present invention relates to a composition for animals comprising a combination of one, two or more acid component(s) in combination with a binding agent.

The binding agent of the disclosure is a tannin component, a polygalactose component, or a combination thereof. Further, the present invention relates to the composition for use as an alternative to medicine and in particular for use in preventing and/or treating diarrhoea in an animal and/or for reducing the use of antibiotics in animal farming. Also disclosed is a method of preparing the composition of the present invention.

### BACKGROUND OF THE INVENTION

During farming, effective production is tightly associated to a high feed conversion rate (FCR), i.e., that the amount of feed administered to animals that is effectively translated into growth (weight gain). Usually, disease is associated with weight loss and therefore, several approaches have been developed in the past to prevent disease and obtain increased growth. In 1951, FDA (the US Food and Drug Administration) approved the first antibiotics for use in animal feed, based on studies showing that it helped chickens, pigs and livestock gain weight. Antibiotics have therefore been used as growth promoters in farming for decades, as supplements to the feed and to avoid infections in animal herds.

Some estimates suggest that the amount of antibiotics applied to treat infections and/or to increase weight gain in livestock worldwide is almost double the amount applied to treat infections in humans. A consequence of this widespread use is the emergence of antibiotic resistant pathogenic bacteria. Antibiotic resistant pathogenic bacteria can infect humans. Increasing cases of multi-resistant bacteria resulted in a plan outlined by FDA to phase out the use of more than 200 antibiotics in farming.

Similarly, the EU decided to ban the use of antibiotics for growth in livestock in 2006. Today, the use of antibiotics in farming is limited to combat infections in herds. With the growing concerns of the increase in antimicrobial resistance, the use of antibiotics has become more tightly controlled in veterinary medicine.

In pig farming, a major infectious disease is diarrhoea. To date, treatment of diarrhoea is most effective using antibiotics. Diarrhoea in piglets is mainly caused by enterotoxigenic Escherichia coli, Campylobacter or Salmonella infections at the neonatal and post-weaning stages—i.e. the first weeks after birth. The highest risk of infection that leads to diarrhoea is at weaning (i.e., when the piglet is separated from its mother). Weaning takes place typically at week 3-5 after birth. At this time, piglets are subjected to many different types of stress, while their gastrointestinal tract is not fully developed. It takes several weeks for a piglet to develop a mature immune system. Together with the transition from the sow's colostrum and its contribution as a passive immune defence to corn-based feedstuff, piglets are at a high risk of infection. When diarrhoea is detected in a farm, treatment of the affected animals or the whole herd is performed using antibiotics.

As an alternative to the use of antibiotics to prevent infection, the use of combinations of pharmaceutical agents (e.g., quinocetone, gamma-aminobutyric acid, sweeteners, nano montmorillonite) has been tested.

Another method to reduce diarrhoea in pigs is the use of zinc. Zinc oxide (ZnO) in therapeutic concentrations (medical zinc oxide) has been widely used to prevent porcine colibacillosis, improve suboptimal weight gain and feed efficiency. The small-intestinal mucosa is altered by feeding of 3000 ppm Zn for 14 days, increasing the absorptive capacity of the small intestine and consequently improving growth. However, the use of zinc in farming is associated with significant environmental burden. The use of zinc in farming was phased out and stopped completely in the EU in 2022.

This ban is likely to an increase the need of antibiotics to treat diarrhoea in pig farming.

As described above, there are currently no sustainable and effective alternatives to the use of antibiotics or zinc in farming. A sustainable alternative will trigger both an economical and a political drive for implementation in farming. Such an alternative will be optimal if it also results in growth promotion without a risk for further development of antibiotic resistance.

Clearly, there is a need in the art to develop a composition, such as a feed additive, a feed composition or a pre-mix that may increase the FCR in animals such as piglets. Also, there is an urgent need for a composition that can prevent and/or treat diarrhoea while simultaneously precluding the use of antibiotics in animals such as piglets. A composition that addresses both of these issues would alleviate the two most prominent problems faced by animal farmers; to reduce or eliminate the use of antibiotics and medical zinc oxide while maintaining or increasing the FCR.

### SUMMARY OF THE INVENTION

The present invention relates to a composition for animals capable of solving the problems in the art—namely to reduce or eliminate the use of antibiotics and/or medical zinc in e.g the agricultural farming industry while maintaining or improving biophysical characteristics such as FCR, weight, drinking water consumption, dry matter content of faeces and/or decrease in symptoms of diarrhoea (such as post weaning diarrhoea (PWD)) of an animal.

Thus, the disclosure relates to a composition comprising an acid component and a binding agent, wherein preferably the binding agent is an E.coli binding agent.

The composition of the disclosure may comprise two or more acid components in combination with a binding agent, wherein preferably the binding agent is an E.coli binding agent.

The composition of the disclosure may comprise an acid component and binding agent, wherein the binding agent comprises a tannin component.

The composition of the disclosure may comprise an acid component and a binding agent, wherein the binding agent comprises a polygalactose component.

The composition of the disclosure may comprise an acid component and a binding agent, wherein the binding agent comprises a tannin component and a polygalactose component.

The weight ratio of acid component to binding agent may be 1-150:1-20; for instance, 1-100:1-20; 1-50:1-20; 5-50:1-20; 5-50:1-10; 5-50:1-5; 5-50:1-2. Preferably the weight ratio of acid component to binding agent is approximately 5-50:1.

The composition of the disclosure may comprise an acid component, a binding agent and a liquid, wherein preferably the liquid is drinking water.

The composition of the disclosure may comprise an acid component, a binding agent and a liquid, wherein preferably the liquid is drinking water, and wherein the pH of the composition is 1 and 7, such as from 2 to 6, e.g. from 2 to 5 and preferably from 2 to 4. Most preferably, the composition for administration has a pH from 2.5 to 3.6, even more preferably from 2.6 to 3.4.

### FIGURES

FIG. 1: Timeline for the challenge experiment set up for experiments wherein the composition comprised lactic acid, acetic acid and tannic acid. The challenge experiment timeline for experiments wherein the composition comprised lactic acid, acetic acid, tannic acid and polygalactose is identical, except that the piglets were only challenged with E. coli on day 1 and day 2.

FIG. 2: Correlation between the pH and different dilutions of the drinking water comprising lactic acid, acetic acid and tannic acid diluted with tap water.

FIG. 3: Quantification of F18 E. coli cells in faeces collected throughout the experiment. The number of F18 E. coli cells was determined by real time PCR and normalized to the amount of starting material (E. coli cfu/g faeces). Results are given as mean values for the test and control groups.

FIG. 4: Development in dry matter content in faeces from test and control group piglets challenged with 10¹⁰ F18 E. coli after weaning. Test piglets received a continuous supply of a composition comprising lactic acid, acetic acid and tannic acid in drinking water from Day 0. Faeces samples collected throughout the experiment were used for dry matter quantification. Dry matter content is for each time point normalized to that observed for each pig at Day 0 zero.

FIG. 5: Faeces score in the pen throughout the challenge experiment as evaluated by an experienced animal keeper. Faeces classification is based on an established continuous scale of four levels (Lee et al. 2017). 1, normal faeces; 2, soft faeces; 3, mild diarrhoea and 4, severe diarrhoea. The faeces score at Day 0 for each group has been subtracted from the values obtained for each day throughout the experiment.

FIG. 6: Percentage of pigs suffering from diarrhea throughout a challenge experiment as evaluated by an experienced animal keeper.

FIG. 7: Feed consumption for test group piglets and control group piglets. Feed consumption is seen to be higher for the test group piglets at all time points and consumption is higher during the period of time where piglets are most severely challenged from the F18 exposure (Day 3 to Day 8 for test group piglets and days 3 to 11 for control group piglets). Growth rate is seen also to increase earlier in test group piglets (starting at Day 9) compared to control pigs (starting at Day 13).

FIG. 8: Feed consumption for test group piglets and control group piglets. Feed consumption is seen to be higher for the test group piglets at most time points.

FIG. 9a: Accumulated water consumption during the challenge experiment for all test group piglets and all control group piglets.

FIG. 9b: Water consumption during the challenge experiment per day per piglet, for test group piglets and control group piglets.

FIG. 10: Water intake during the challenge experiment per day per piglet, for test group piglets and control group piglets.

FIG. 11: Weight gain of piglets being infected with F18 ETEC according to the timeline in FIG. 1. From Day 0 the test group piglets had unlimited access to drinking water supplemented with lactic acid, acetic acid and tannic acid, while the control group piglets had unlimited access to tap water.

FIG. 12: Weight gain of piglets being infected with F18 ETEC according to the timeline in FIG. 1. From Day 0 the test group piglets had unlimited access to drinking water supplemented with lactic acid, acetic acid, tannic acid and polygalactose, while the control group piglets had unlimited access to tap water.

FIG. 13: Results of the E. coli agglutination test. From left to right, images of droplets of E. coli cells that have been mixed with (i) lactic acid and acetic acid, (ii) tannic acid, and (iii) lactic acid, acetic acid and tannic acid on a glass slide.

FIG. 14: Results of the E. coli agglutination test. Panel A shows droplets of E. coli cells that have been mixed with NaCl solution, while panel B shows droplets of E. coli cells that have been mixed with lactic acid, acetic acid, tannic acid and polygalactose. This combination is shown to cause agglutination.

FIG. 15: Results of the E. coli agglutination test. Panel A shows droplets of E. coli cells that have been mixed with NaCl solution, while panel B shows droplets of E. coli cells that have been mixed with lactic acid and acetic acid. This combination is shown not to cause agglutination.

FIG. 16: Results of the E. coli agglutination test. Panel A shows droplets of E. coli cells that have been mixed with NaCl solution, while panel B shows droplets of E. coli cells that have been mixed with tannic acid and polygalactose at a pH of 2.7 as adjusted with HCl. This combination is shown to cause agglutination.

FIG. 17: Results of the E. coli agglutination test. Panel A shows droplets of E. coli cells that have been mixed with NaCl solution, while panel B shows droplets of E. coli cells that have been mixed with tannic acid and polygalactose at a pH of 2.7 as adjusted with H₂SO₄. This combination is shown to cause agglutination.

FIG. 18: Results of the E. coli agglutination test. Panel A shows droplets of E. coli cells that have been mixed with NaCl solution, while panel B shows droplets of E. coli cells that have been mixed with lactic acid, acetic acid and polygalactose. This combination is shown to cause agglutination.

### DETAILED DESCRIPTION OF THE INVENTION

### Definitions

Unless defined otherwise, all technical and scientific terms used herein have the same meaning as commonly understood by one of ordinary skill in the art to which this disclosure belongs.

### An Acid Component

An ‘acid component’ in the context of the present disclosure is a water-soluble acid which is suitable for animal or human consumption.

The acid component is included in order to reduce the pH of the composition. A low pH is beneficial for ensuring that all composition components are soluble when the composition is a liquid phase.

The acid component may also help prevent/improve resistance to sickness and further may enhance the effects of the binding agent.

The acid component of the disclosure may be a combination of one, two or more acids. The acid component, and each acid in the acid component, may have a pKa value from 1 to 7, preferably, between 3 and 5.

Preferably, the acid component is water-soluble.

In the context of the disclosure, ‘water-soluble’ is used to describe a compound that dissolves or disperses in water to provide a clear or transparent liquid, without precipitate. For example, ‘water-soluble’ according to the disclosure describe a compound that produces a clear or transparent liquid without precipitate when 1 g is added to 1 L of water at 25° C.

Suitable acids for the acid component are selected from the list comprising lactic acid, acetic acid, hydrochloric acid, phosphoric acid, nitric acid, sulphuric acid, or combinations thereof.

Preferably the acid component comprises lactic acid. More preferably, the acid component comprises lactic acid and a second acid.

Preferably, the acid component comprises an acid palatable to piglets, for example acetic acid, to ensure piglets consume the composition at high rates.

Preferably the second acid is an acid palatable to piglets. Typically, piglets dislike the taste of lactic acid, and may therefore avoid its consumption. The addition of even small amounts of a second acid can disguise the taste of lactic acid and ensure that the piglets consume the composition at an appropriate rate.

By using a second acid to enhance the organoleptic properties of the composition, flavour enhancers such as sweeteners and sugars can be omitted. Sugar-containing additives typically cause bacterial proliferation in the gastro-intestinal tract, and therefore it is beneficial to provide a composition that is palatable to piglets that does not comprise a sugar component.

The second acid component may also be used to regulate the pH of the composition. Preferably, the second acid is acetic acid, hydrochloric acid, sulphuric acid or combinations thereof. Preferably the second acid is acetic acid.

Lactic Acid ‘Lactic acid’ in the context of the present invention is lactic acid, or a salt or ester thereof. ‘Lactic acid’ further relates to any compound that provides lactic acid, or a variant thereof, in solution. Lactic acid may also be termed C₃H₆O₃ or LA, and has the general chemical formula:

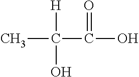

Lactic acid may be in the form of L-(+)-lactic acid (L-Lactic acid) or (S)-lactic acid or as their mirror images, D-(−)-lactic acid (D-Lactic acid) or (R)-lactic acid.

Lactic acid may also be present as a mixture of the two called DL-lactic acid, or racemic lactic acid. Optionally, the lactic acid component is a liquid lactic acid or salt or ester thereof.

Lactic acid is a low pKa acid and is therefore a good pH regulator.

### Binding Agent

The composition of the disclosure comprises a binding agent. The term ‘binding agent’ as used herein refers to a compound that can bind to the fimbriae of pathogenic bacteria, particularly the fimbriae of E.coli bacteria.

The function of the binding agent is inhibiting the attachment of pathogenic bacteria to the intestinal walls and the gastro-intestinal tract.

Many pathogenic bacteria comprise long fibrils called fimbriae via which bacteria attach to the walls if the gastro-intestinal tract. Binding agents according to the invention are able to bind to the fimbriae, for example via hydrogen bonding, ionic bonding and/or van der Waals interactions.

Once a binding agent is bound to fimbriae, bacteria can no longer attach to the intestinal walls and therefore cannot proliferate in the gastro-intestinal tract. This improves resistance to sickness, prevents sickness, and/or prevents the negative side-effects of weaning in animals and humans.

Binding agents according to the disclosure may bind to the tip or along the length of bacterial fimbriae. Preferably, the binding agent is able to bind to both the length and the tip of fimbriae.

The binding agent may be a single component or a mixture of components, wherein one or more components may be responsible for tip and length binding.

Preferably, the binding agent can bind more than one fimbria simultaneously. The more than one fimbria may be on the same bacteria, or they maybe on different bacteria. Without wishing to be bound by theory, binding more than one fimbria simultaneously causes agglutination of pathogenic bacteria and severely inhibits intestinal wall binding.

The binding agent should be present in an amount suitable for binding to fimbriae and preventing adhesion of pathogenic bacterial to the intestinal walls. Preferably the binding agent is present in an amount suitable for causing agglutination. The amount suitable for preventing adhesion/causing agglutination will depend on the nature of the binding agent.

For some bindings agents, the composition conditions may also need to be adjusted to facilitate binding and/or agglutination. Examples of such conditions include a low pH, the presence of a salt, the presence of another binding agent, and, when present, the type and amount of liquid in the composition.

For determining appropriate composition conditions for a given binding agent, an assay may be prepared. Suitable assays include assays for visual monitoring of agglutination properties.

An example of an assay comprises the steps:
Preparing a composition comprising an acid component, a binding agent, and optionally a liquid.
Preparing a bacterial strain comprising fimbriae by washing three times with 0.9% sodium chloride.
Resuspending the washed bacteria cells to obtain an optical density (OD450) of 1.5 in 50 μL 0.9% sodium chloride.
Mixing 5 μL of the cell suspension with 5 μL of either a control composition consisting of 0.9% NaCl or 5 μL of a composition comprising an acid component and binding agent.
Placing a droplet of the mixture on the surface of a glass plate and monitoring visual changes for 3 minutes.
Compositions according to the invention will cause visible agglutination of the pathogenic bacterium over the course of the experiment (the formation of white dots), wherein exemplary compositions will cause immediate visible agglutination.

If agglutination does not occur after 3 minutes, the composition or the conditions may be unsuitable for the object of the invention. If agglutination does not occur, the composition may be adjusted, for example by decreasing the pH (e.g., increase the acid component concentration), increasing the concentration of binding agent, or adjusting the ratio of binding agent components if more than one is present.

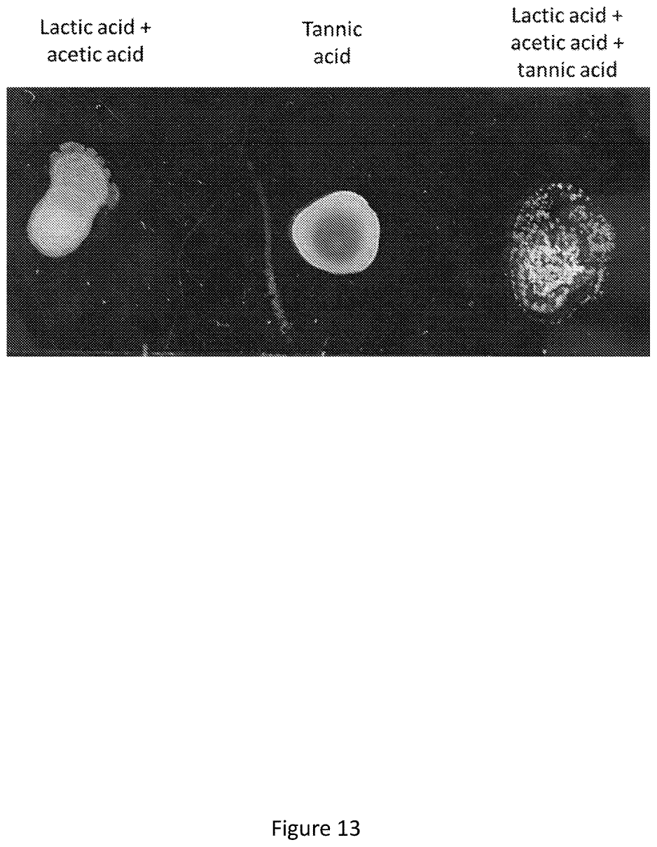

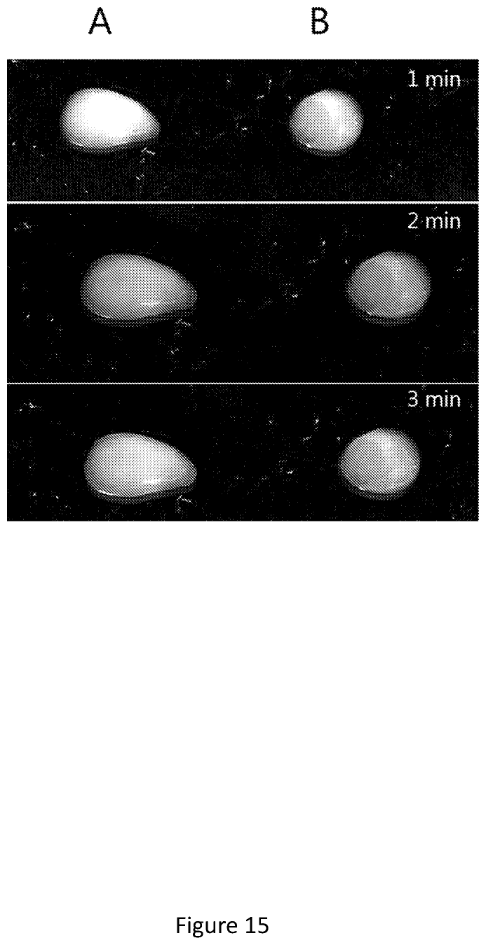

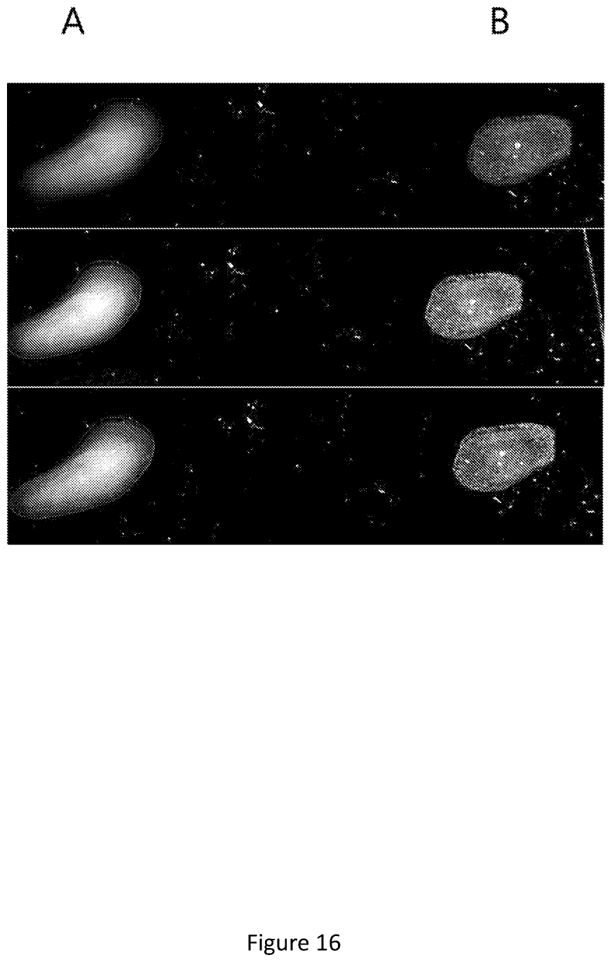

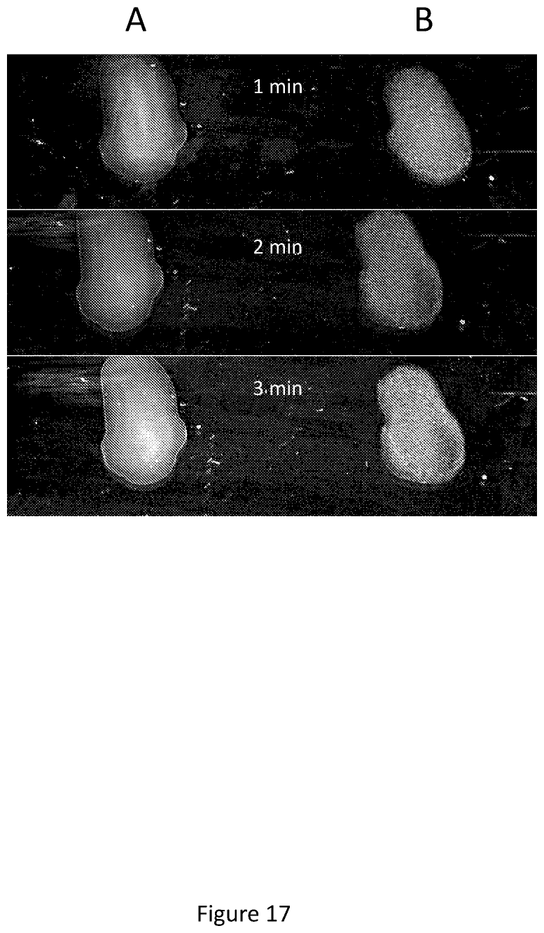

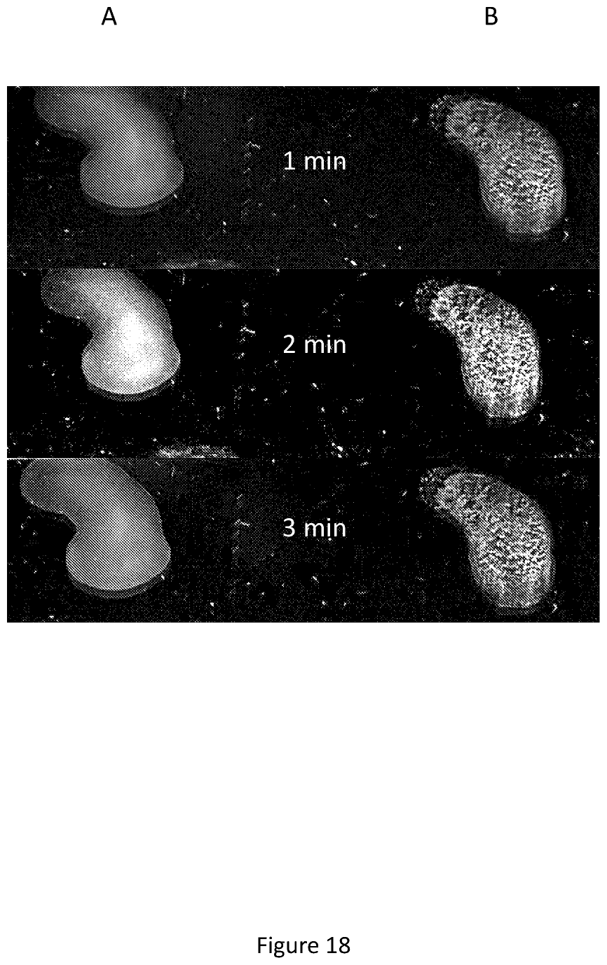

Examples 4.1 and 4.2 and corresponding FIGS. 13-18 show the results of testing different compositions and composition conditions according to the above assay. Examples of agglutination can be seen in FIGS. 13, 14, 16, 17 and 18.

Preferably, the binding agent of the disclosure is soluble in water, even more preferably the binding agent of the disclosure is soluble in water at pH 7 or below, for example at pH 4 or below.

Preferably, the binding agent is soluble in water at a pH from 1 to 7, such as from 2 to 6, e.g. from 2 to 5 and preferably from 2 to 4. Most preferably, the composition for administration has a pH from 2.5 to 3.6, even more preferably from 2.6 to 3.4.

Exemplary binding agents according to the disclosure are E-coli binding agents. Examples of suitable E. coli binding agents include tannin components, polygalactose components, or combinations thereof.

### A Tannin Component

The binding agent of disclosure may comprise a tannin component.

The term ‘tannin component’ as used herein refers to polyphenolic compound or the salt or ester thereof. Typically, tannin components comprise a carbohydrate centre surrounded by phenol-containing moieties.

The phenol-containing moieties are typically galloyl moieties or their derivatives, which are bound to the central carbohydrate moiety through ester linkages.

The hydroxyl groups of the central carbohydrate may be partially or totally esterified with galloyl moieties or their derivatives to form said ester linkages. Often the central carbohydrate is glucose, while the galloyl moity is based on gallic acid.

Preferably, the central carbohydrate group is functionalized with galloyl moieties in more than on location, for example, two, three, four or more locations.

Preferably, the galloyl moiety is distanced from the central carbohydrate by a spacer group. The spacer group may be a galloyl moiety or derivative thereof, or the spacer group may be any other suitable moiety.

As used herein, a ‘tannin component’ is particularly used to refer to a carbohydrate that has two or more of its hydroxyl groups bound via ester linkages to galloyl moieties.

Tannins may be extracted from plants, or be a plant metabolite, and as such a variety of degrees of functionalization is seen resulting in the weight distribution of tannins. The tannin component may therefore weigh between 300 Da and 3000 Da. For example, between 500 Da and 2000 Da. Preferably, between 1200 Da and 1900 Da.

An exemplary tannin component is tannic acid, or the salt or ester thereof.

Preferably the tannin component of the invention is tannic acid. Tannic acid may also be termed 1,2,3,4,6-penta-O-{3,4-dihydroxy-5-[(3,4,5-trihydroxybenzoyl)oxy]benzoyl}-D-glucopyranose, C₇₆H₅₂O₄ or TA and has the chemical formula:

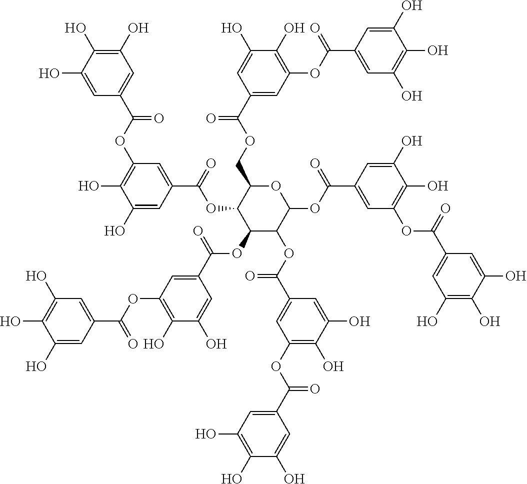 [SMILES: O=C(OC[C@H]1OC(OC(=O)c2cc(O)c(O)c(OC(=O)c3cc(O)c(O)c(O)c3)c2)C(OC(=O)c2cc(O)c(O)c(OC(=O)c3cc(O)c(O)c(O)c3)c2)[C@@H](OC(=O)c2cc(O)c(O)c(OC(=O)c3cc(O)c(O)c(O)c3)c2)[C@@H]1OC(=O)c1cc(O)c(O)c(OC(=O)c2cc(O)c(O)c(O)c2)c1)c1cc(O)c(O)c(OC(=O)c2cc(O)c(O)c(O)c2)c1]

Typically, tannin components according to the disclosure bind fimbriae by forming hydrogen bonds along the length of the fimbriae. This inhibits pathogenic bacterial, such as E-coli, from binding to the intestinal wall via the lengths.

Due to the size and geometry of the tannin component, it can bind multiple fimbriae simultaneously, wherein the fimbriae are either neighbouring fimbriae on the same bacteria or are fimbriae from two different bacteria, and can thus cause agglutination. Multi-site binding allows for faster and/or more agglutination of the bacteria, further inhibiting its ability to bind to the intestine. Therefore, it is preferably that the tannin component has a plurality of binding sites so that it can bind E. coli at two, three, four, five or more sites simultaneously.

Tannic acid or a salt or ester thereof and albumin tannate are both ‘tannins’. Tannic acid or a salt or ester thereof has more free binding sites than albumin-tannate and thus it is considered that tannic acid is more effective than albumin-tannate at binding pathogenic bacteria and inhibiting growth. Of the two, only tannic acid is a tannin component according to the invention.

Preferably, tannin compounds according to the disclosure are water-soluble, and in particular are water-soluble at low pH.

### A Polygalactose Component

The binding agent of the disclosure may comprise a polygalactose component. The term ‘polygalactose component’ as used herein refers to a water-soluble polymer of galactose, a galactose derivative, or a salt or ester thereof.

The polygalactose component of the disclosure is a polysaccharide. Polysaccharides are much larger polymers than oligosaccharides—typically oligosaccharides comprise 3-15 monosaccharide sub-units whilst polysaccharides comprise 16 or more monosaccharide sub-units. In the context of the disclosure, polygalactose components comprising more than 16 monosaccharide sub-units are considered polysaccharides.

The polymer can be a linear or a branched polysaccharide. Preferably, the polygalactose component is branched.

Polysaccharides often have poor solubility in water. Preferably, the polymer contains moieties that improve water-solubility. Exemplary water-solubilising moieties include sulphate, which for instance may be bonded to one of the hydroxyl groups on the galactose moiety.

The polygalactose component may therefore be a sulphated polygalactose.

Exemplary sulphated polygalactoses include carrageenans, with kappa-carrageenan being particularly preferred.

Typically, the weight average molecular weight of the polygalactose component is between 10,000 Da and 1,000,000 Da, such as between 50,000 Da and 75,000 Da. Preferably the molecular weight is between 150,000 Da and 300,000 Da. The weight average molecular weight may be determined by size exclusion column chromatography coupled with multi-angled laser light scattering, commonly referred to as SEC-MALLS (or SEC-MALS). Any suitable SEC method may be used, for example gel permeation chromatography (GPC).

The SEC column serves to separate molecules by hydrodynamic volume. After exiting the column of the HPLC or FPLC system, the molecules pass through a MALS detector and are probed by a laser beam. The MALS signals, along with UV absorbance and/or differential refractive index (dRI) signals, are analyzed to quantify the analyte's physical properties.

In an example method, the weight average molecular weight may be determined via GPC performed on a Shimadzu Prominence LC system equipped with an RI detector with a 300 mm×75 mm, 5 μm PLgel 100 Å and 300 mm×7.5 mm, 5 μm PLgel 500 Å column in series at 40° C. with an eluent rate of 1.0 ml/min. The method may be calibrated with poly(styrene) standards with MW between 1000 and 10000. The sample may then be passed through an Wyatt DAWN Heleos II MALLS detector and a Wyatt Optilab T-rEX RI detector.

The binding interaction between polygalactose and fimbriae may be different depending on the units of which the fimbriae are comprised.

Typically, polygalactose components according to the disclosure bind to the tips of fimbriae. Binding the fimbriae with polygalactose may therefore inhibit bacteria from binding to the intestinal wall via end groups.

The polygalactose of the disclosure may also bind along the length of the fimbriae, by binding of the polygalactose to the fimbrial sub-unit.

Again, the size and geometry of the large polysaccharide polylgalactose allows for multi-site binding, and one polygalactose component can bind multiple fimbriae, either from the same bacteria or no different bacteria, and thus may cause agglutination. This may again facilitate faster and/or more agglutination of the bacteria, providing exemplary results. It is therefore preferably that the polygalactose component has a plurality of binding sites so that it can bind E. coli at two, three, four, five or more sites simultaneously.

Typically, polygalactose binding agents require a low pH in order to facilitate agglutination of pathogenic bacteria. For example, a pH of less than 7, less than 4 or less than 3 may be appropriate. For example, a pH from 1 to 7, such as from 2 to 6, e.g. from 2 to 5 and preferably from 2 to 4. Most preferably, the composition for administration has a pH from 2.5 to 3.6, even more preferably from 2.6 to 3.4.

The pH-sensitivity of polygalactose may depend on the presence of other binding agents in the composition, or the nature of the liquid, when present.

### Liquid

The term ‘liquid’ or ‘liquid composition’ according to the present invention relates to compositions that are wet, liquid, or semi-liquid, wherein a wet or semi-liquid composition is a composition that has a consistency between a dry composition and a liquid composition.

In the context of the invention, a dry feed has a liquid content of 10% or less, a liquid feed has a content of 75% or more, and a wet composition or a semi-liquid composition has a liquid content of between 10% and 75%.

A liquid composition may comprise any liquid that is suitable for animal or human consumption. Preferably, the liquid is water, such as drinking water.

Preferably, the components of the composition are soluble in the liquid at the quantities required for preventing adhesion of bacteria to the intestinal wall.

Typically, a liquid composition according to the disclosure is acidic and has a low pH, for example below pH 7 due to the presence of the acid component.

Preferably, a liquid composition according to the disclosure has a pH from 1 to 7, such as from 2 to 6, e.g. from 2 to 5 and preferably from 2 to 4. Most preferably, the composition for administration has a pH from 2.5 to 3.6, even more preferably from 2.6 to 3.4.

### Weaning

The term ‘weaning’ relates to the process of accustoming a young mammal to food other than milk, for example it's mother's milk. Commonly, weaning occurs at pig farms when piglets are from 2 to 5 weeks old, and can take place over a period of between 2 and 8 weeks.

In some cases, piglets may be weaned directly from milk to dry feed, and in other cases piglets may be weaned gradually and are offered both milk and dry feed over a transition period. During the transition period, piglets may also be offered a wet feed, a semi-liquid feed or a liquid feed.

In the examples of the invention, piglets are weaned directly from milk to dry feed and ‘weaning’ occurs on the first day that piglets do not receive milk (Day 0 in the Examples of the application).

The term ‘post-weaning’ relates to the period after weaning has begun. As such, the terms ‘weaning’ and ‘post-weaning’ may be used interchangeably in the present application.

### Zinc, Medical Zinc, Additive Zinc

The terms ‘zinc’, ‘medical zinc’ or ‘additive zinc’ relate to an amount of zinc included in a piglet's diet in order to treat or prevent sickness. The zinc may be administered via a feed, feed additive or other composition, and is often administered as zinc oxide (ZnO). The terms ‘zinc, ‘medical zinc’ and ‘additive zinc’ therefore also encompass ‘zinc oxide’, ‘medical zinc oxide’ and ‘additive zinc oxide’.

The terms ‘zinc’, ‘medical zinc or ‘additive zinc’ as used herein refer to an amount of zinc that is greater than the minimum amount of zinc required in pig feed to maintain biophysical characteristics.

Typically, around 120-130 mg of zinc per kg composition, and up to a maximum of 150 mg zinc per kg of composition, are used for maintaining normal physiological conditions in pigs. In the context of the invention, a composition or feed comprising ‘zinc’, ‘medical zinc’ or ‘additive zinc’ is therefore a composition that comprises more than 150 mg zinc per kg composition. Often, a feed comprising ‘medical zinc’ comprises around 1.5-2.5 g zinc per kg composition.

Free from Medical Zinc

On the other hand, a composition that does not comprise more than 150 mg zinc per kg is a composition that is ‘free from medical zinc’. A composition ‘free from medical zinc’ is therefore a composition that does not include zinc for the purpose of treating or preventing sickness.

### Feed

The term ‘feed’ or ‘feed composition’ used herein relates to a composition for feeding animals, preferably for feeding piglets.

The feed may comprise wheat, barley, soybean meal, soybean concentrate, whey powder, potato protein, soybean oil, fish meal, vitamins, and minerals. The feed may or may not comprise medical zinc.

The feed may be a dry feed, a wet feed, a liquid feed, or a semi-liquid feed. The feed may be in the form of granules, powder, pellets, flakes, a pressed feed, or another form.

### Feed Additive

The term ‘feed additive’ or ‘feed additive composition’ relates to a composition provided to animals in addition to standard intake of feed, for example, a dry feed. The feed additive may be incorporated in a standard feed; added to a standard feed; or be administered separately to a standard feed, either at the same time or at different times. The feed additive composition of the invention may be a dry feed additive, a wet feed additive, a liquid feed additive, or a semi-liquid feed additive.

### Pre-mix

The term ‘pre-mix’ as used herein relates to pre-mixes, precursors, base mixes, mash, and diluents for pellets. Pre-mixes typically comprise vitamins and trace minerals. Pre-mixes may also comprise food and animal feed ingredients such as dicalcium phosphate, limestone, salt and a vitamin and mineral pre-mix, but not grains and protein ingredients. Diluents include, but are not limited to, grains (for example wheat middlings and rice bran) and clays, such as phyllosilicates (the magnesium silicate sepiolite, bentonite, kaolin, montmorillonite, hectorite, saponite, beidellite, attapulgite, and stevensite). Clays also function as carriers and fluidizing agent, or diluents, for food and animal feed premixes. Mash typically comprises a complete animal diet. For example, the mash comprises or consists of corn, soybean meal, soy oil, salt, DL Methionine, limestone, dicalcium phosphate and vitamins and minerals.

### Post-Weaning Diarrhoea (PWD)

In the early stages of weaning, colostrogenic immunity declines and lactogenic immunity is lost due to discontinued suckling. Piglets are commonly weaned when they are around 2-5 weeks old, and at this stage, have an immature gut with a sub-optimal microbial community, an undeveloped mucosal-immunity and an impaired intestinal epithelial barrier function that is not adapted to solid feed. In addition, the gut health might be further compromised by the stressful events during weaning, and the new environment where surface contamination and pen mates might introduce the piglets to new pathogens. This can lead to post-weaning diarrhoea.

Post-weaning diarrhoea (PWD) refers to a multifactorial condition occurring in the first 14 days after weaning where the clinical signs of diarrhoea occur. Clinical signs of diarrhoea include defecation with increased rate, volume and water content, or defecation with a decreased dry matter content.

This can occur due to the aforementioned factors or due to infection from pathogens. Examples of pathogens known to cause post-weaning diarrhoea include entero toxigenetic E coli bacteria, rotavirus, and porcine enteric coronaviruses. One of the most common entero toxigenetic bacteria that causes PWD is Escherichia coli (ETEC).

PWD with no detection of ETEC is also common, and it has been suggested that an intestinal dysbiosis in itself might cause intestinal inflammation and diarrhoea after weaning in pigs.

### Symptoms of PWD

Symptoms of PWD include, but are not limited to, diarrhoea, an increased defecation amount, an increased defecation frequency, a decreased faeces dry matter content, a loss of appetite, a reduced feed or drinking water intake, reduced weight gain and/or weight loss.

### Biophysical Characteristics

The term ‘biophysical characteristics’ as used herein relates to one or more of: weight; weight gain; mass; height; growth; growth rate; body fat percentage, body fat distribution, egg size; egg weight; egg mass; egg laying rate; mineral absorption; mineral excretion, mineral retention; bone density; bone strength; feed conversion ratio; feed conversion rate; drinking water consumption; feed consumption; dry matter content of faeces; retention of any one or more of copper, sodium, phosphorous, nitrogen and calcium; amino acid retention or absorption; mineralization and bone mineralization; symptoms of post-weaning diarrhoea.

Accordingly, the term ‘improved biophysical characteristics’ relates to an improvement or increase in one or more biophysical characteristics of the aforementioned list, with the exception of symptoms of post-weaning diarrhoea, wherein a decrease relates to ‘improved biophysical characteristics’.

Preferably, ‘improved biophysical characteristics’ refers to an increase in one or more of: feed conversion ratio; body weight; drinking water consumption; feed consumption; dry matter content of faeces; or a decrease in symptoms of post-weaning diarrhoea.

### Feed Conversion Ratio

“Feed conversion ratio” (FCR) as used herein, refers to the amount of feed fed to an animal to increase the weight of the animal by a specified amount.

This is a measure of feed efficiency and is calculated as follows:

FCR(Feed Conversion Ratio)=FI/BWG

where FI=feed intake of the animal over a specified time period and BWG=gain in body weight over the same period.

BWG is calculated by subtracting the start weight of the animal from the weight of the animal at the end of a defined period e.g., a trial. The feed conversion ratio is essentially how many kilograms of feed needs to be consumed per kg of weight gain. This is calculated by taking the total amount of feed consumed (in kg, either for a given period or over the whole trial) and dividing it by the bodyweight gain (in kg, either for the given period or over the whole trial).

An improved feed conversion ratio means a lower feed conversion ratio.

By “lower feed conversion ratio” or “improved feed conversion ratio” it is meant that the use of the composition of the invention results in a lower amount of feed being required to increase the weight of the animal by a specified amount, compared to the amount of feed required to increase the weight of an animal that is fed without use of the inventive composition.

For farmed pigs it contemplated that the FCR is around 3.1. As can be seen in the examples the FCR for control pigs are around 2.3 whereas the FCR for test pigs are 1.6.

### Drinking Water

In the context of the invention, ‘drinking water’ relates to potable water offered to animals.

In the context of the invention, the term ‘tap water’ relates to potable water offered to animals that specifically does not comprise the composition of the invention.

### Dry Matter Content

The term ‘dry matter content’ as used herein relates to the dry matter content of faeces. The term ‘faecal dry matter content’ may also be used.

Dry matter content in faeces is a universally accepted indicator of the extent of diarrhoea. For determination of dry matter content, faeces were collected from the pens, and weighed. The collected samples were freeze dried and the water content was determined as mass before freeze drying subtracted mass after freeze drying.

diarrhoea Dry matter percentage=100×(mass after water evaporation by freeze drying/mass before water evaporation).

A low dry matter content is indicative of diarrhoea.

### Pathogenic Bacteria

The term ‘pathogenic bacteria’ relates to bacteria that can cause disease, for example bacteria that can cause disease in swine.

Examples of pathogenic bacteria according to the invention are entero toxigenetic E coli bacteria, rotavirus, and porcine enteric coronaviruses. Examples of entero toxigenetic bacteria are Escherichia coli (ETEC) bacteria such as F4 or F18. The enterotoxigenic ETEC expressing F4 and F18 fimbriae are the two main pathogens associated with post-weaning diarrhea (PWD) in piglets. E. coli F4 is to be understood as an E. coli expressing F4 fimbriae whereas E. coli F18 is to be understood as an E. coli expressing F18 fimbriae.

### Resistance, Tolerance

The present invention relates to increasing the subject's ‘resistance’ to entero toxigenetic E coli bacteria. In other words, the present invention relates to avoiding or reducing the negative effect of entero toxigenetic E. coli bacteria.

The term ‘resistance to’ as used herein may encompass the term ‘tolerance of’. Therefore in one embodiment the subject may not be resistant to entero toxigenetic E coli bacteria but the subject may be able to tolerate the entero toxigenetic E coli bacteria, i.e. without or with reduced negative effects on the biophysical characteristics of the subject.

Preventing growth of entero toxigenetic E coli bacteria in the gastrointestinal tract, or a decrease in the population of entero toxigenetic E coli bacteria in the gastrointestinal tract are examples of ‘resistance to’ entero toxigenetic E coli bacteria.

### Weight Gain

The present invention relates a method and/or use of a composition for increasing weight gain in a subject, e.g. a piglet, comprising feeding and/or watering the subject with a composition according to the present invention.

An ‘increased weight gain’ refers to an animal having increased body weight when fed with a composition comprising the composition of the invention compared with an animal that is fed with a composition that does not comprise the composition of the invention.

### Average Weight Gain

The average weight gain (AWG) for a group of piglets is the total weight gained by the group divided by the number of piglets in the group.

### An Animal in Need Thereof

In the context of the invention, ‘an animal in need thereof’ is an animal that requires feeding, preferably a piglet, more preferably a weaning or post-weaning piglet.

The ‘animal in need thereof’ may or may not show symptoms of PWD.

### Feeding an Animal

The term ‘feeding an animal’ as used herein relates to providing an animal with a feed composition and/or water. The term ‘feeding an animal’ may therefore also encompasses ‘watering an animal’.

pH

The ‘pH’ of a composition, as referred to herein, is the pH at a temperature of between 20-25° C. The pH can be measured via a pH strip such as litmus paper, via a pH meter, or another recognized method.

Preferably the pH is measured with a pH meter, for example Thermo Scientific™ Orion Star™ A211 Benchtop pH Meter.

### Mineral

In the present context and in the context of nutrition a ‘Mineral’ is to be understood as a chemical element required as an essential nutrient by organisms to perform functions necessary for life. A mineral can be selected from the group consisting of calcium, phosphorus, potassium, sodium, magnesium, sulphur, iron, chlorine, cobalt, copper, zinc, manganese, molybdenum, iodine, selenium, and a combination thereof.

### Vitamin

In the present context the term ‘Vitamin’ is to be understood as an organic molecule that is an essential micronutrient which an organism needs in small quantities for the proper functioning of its metabolism. A vitamin can be selected from the group consisting of vitamin A, vitamin B1, vitamin B2, vitamin B3, vitamin B5, vitamin B6, vitamin B7, vitamin B9, vitamin B12, vitamin C, vitamin D, vitamin E, vitamin K and any combination thereof.

### DETAILED DESCRIPTION

The inventors of the present invention have among others developed a composition for increasing weight gain in post-weaning piglets. Fast weight gain in piglets is beneficial to pig farmers in order to maximize FCR (feed conversion ratio), but also to increase the piglet's resistance to sickness. It is commonly known that the likelihood of sickness in piglets is decreased as their weight increases.

The composition of the present invention can also be used to prevent and/or treat sickness in post-weaning piglets. For example, the composition of the present invention can prevent sickness by reducing the growth of pathogenic bacteria in the gastrointestinal tract which can lead to diarrhoea, or specifically post-weaning diarrhoea (PWD) in the case of post-weaning piglets. Zinc is widely used in piglet feed in order to prevent and/or treat the symptoms of PWD, however the use of zinc, for example zinc oxide, has been phased out in the European Union due to health and pollution concerns. The composition of the invention can therefore be used as an alternative to zinc oxide in piglet feed to improve the biophysical characteristic of post-weaning piglets.

Similarly, the composition may be used to reduce and or eliminate the need for antibiotics in pig farming. Antibiotics are currently used to prevent and/or treat PWD and avoid mortality in piglets. Widespread use of antibiotics is unfavourable due to the risk of antibiotic resistance.

The composition of the invention therefore has clear advantages over the use of zinc, antibiotics and/or other growth promoters in pig farming. Numerous attempts have been made to develop a solution to phase out medical zinc in pig farming without increasing the need for antibiotics. These attempts have been unsuccessful, and it is therefore very surprising that the inventors of the present invention succeeded in developing a composition addressing and solving these problems and even more surprisingly while observing a weight increase in the animals when comparing to a control group of animals that did not receive the composition. A further advantage of the composition of the present invention that makes the invention highly commercially interesting is that it is inexpensive and easy to administer. Farmers are traditionally very reluctant changing their animal feed and it's therefore an additional advantage that the farmer can continue applying the feed preferred since the composition of the present invention can be administered directly in the drinking water or alternatively poured over the feed prefeed by the farmer.

Thus, the disclosure relates to a composition for animals comprising acid component(s) in combination with a binding agent.

In the composition, the ratio of acid component to binding agent may be 1-150:1-20; for instance, 1-100:1-20; 1-50:1-20; 5-50:1-20; 5-50:1-10; 5-50:1-5; 5-50:1-2. Preferably the weight ratio of acid component to binding agent is approximately 5-50:1.

In an embodiment, the composition comprises 70-99.9% (w/w) acid component and 0.1-30% (w/w) binding agent.

For instance, the composition may comprise 72-99.5% (w/w) acid component, for instance 75-99% (w/w), 80-99% (w/w) or 82-98% (w/w) acid component.

The composition may comprise 0.5-28% (w/w) binding agent, for instance 1-25% (w/w), 1-20% (w/w), or 2-18% (w/w) binding agent.

In some embodiment, the composition comprises the acid component and the binding agent.

In some embodiments, the composition consists of the acid component and the binding agent.

In a preferred embodiment, the acid component(s) comprises lactic acid and/or the ester or the salt.

In an embodiment the acid component(s) comprises lactic acid and a second acid. This combination increases the organoleptic properties to make the composition more attractive for animals to drink and/or eat. However, other combinations may have similar organoleptic properties.

Preferably in such embodiments, the weight ratio second acid:lactic acid is in the range of 1-5:20-200. Preferably, the weight ratio is approximately 1:50.

In an embodiment, the acid component(s) comprises lactic acid and a second acid, wherein the weight ratio of second acid:lactic acid is in the range of 1-5:20-150, such as 1-5:25-100, e.g. 1-5:25-75, such as 1-2:25-60.

The above weight ratios correspond to appropriate weight ratios to improve the organoleptic properties when the second acid is acetic acid. When the second acid is an acid other than acetic acid, or the acid component does not comprise lactic acid, different weight ratios may be appropriate.

In some embodiments, the binding agent comprises a tannin component.

In another embodiment the composition comprises an acid component(s) and a tannin component and wherein the weight ratio of acid component:tannin component is in the range of 2-200:1, preferably the weight ratio is 2-100:1, for instance 2-50:1, 5-40:1, or 5-30:1.

Preferably, the tannin component is a tannic acid or a salt or ester thereof.

In an embodiment, the composition comprises 72-99% (w/w) acid component, for instance 75-99% (w/w), 80-99% (w/w) or 82-98% (w/w) acid component and 1-20% (w/w) tannin component, for instance 2-18% (w/w), 2-15% (w/w), or 3-13% (w/w). In a preferred embodiment, the composition comprises around 87-96% (w/w) acid component and around 3-13% (w/w) tannin component.

In some embodiments, the binding agent comprises a polygalactose component.

Polygalactose is an exemplary binding agent and thus only very small quantities are required for obtaining the desired effects.

In an embodiment the composition comprises acid component(s) and a polygalactose component, wherein the weight ratio acid component(s):polygalactose component is in the range of 5-350:1, preferably the weight ratio is 20-300:1, for instance 50-300:1, 100-300:1, or 200-300:1.

In an embodiment, the composition comprises 72-99% (w/w) acid component, for instance 75-99% (w/w), 80-99% (w/w) or 82-98% (w/w) acid component and, 0.01-5% (w/w) polygalactose component, for instance 0.01-3% (w/w), 0.03-2% (w/w), 0.05-2% (w/w), 0.1-2% (w/w), or 0.2-1% (w/w) % polygalactose component.

In an embodiment, the binding agent comprises a tannin component and a polygalactose component.

Tannin and polygalactose components bind to different parts of the fimbriae, and as such the combination of the two is considered particularly advantageous, and may even provide a synergistic effect.

Thus, in an embodiment the composition comprises an acid component(s), a tannin component and a polygalactose component, wherein the weight ratio acid component(s):tannin component:polygalactose component is in the range of 100-300:5-20:1, for instance 200-300: 5-15:1, 250-300:8-12:1. Preferably, the weight ratio of acid component(s):tannin component:polygalactose component is approximately 269:9.5:1.

In an embodiment, the composition comprises about 95% (w/w) acid component, about 3.4% (w/w) tannin component and about 0.35% (w/w) polygalactose.

The amount and/or weight ratio of binding agent in the composition should be sufficient to bind the fimbriae of pathogenic bacteria and obtain the desired result. Preferably, the amount and/or weight ratio of binding agent is sufficient to cause agglutination of pathogenic bacteria.

The composition of the disclosure may be diluted for storage, transport and/or administration. Preferably, when diluted, the composition is diluted with water for form a liquid composition.

In some embodiments, the composition is diluted 1×, 2×, 3×, 10× or 20× times by weight to form a liquid composition suitable for storage and/or transport. Such a composition may be referred to as a ‘composition for storage/transport’ herein. A composition for storage/transport remains a composition according to the invention.

In embodiments wherein the composition is diluted with water, the amount of water should be such that the acid components(s) and the binding agent(s) are solubilized. Preferably, a composition for storage/transport is diluted with the minimum amount of water to solubilize all components in order to avoid storage and transport of excess water.

Some binding agents, for example polygalactose, are difficult to solubilize and as such, sufficient water should be added to the composition in order to solubilize polygalactose in embodiments wherein it is present. For example, a composition for storage and/or transport comprising polygalactose may comprise up to 20%, 30%, 40% or 50% (w/w) water.

When all components are in solution, the liquid composition is a clear liquid. When one or more components is not in solution, the liquid composition may be cloudy or opaque.

A cloudy liquid composition may become clear over time as the components solubilize. Accordingly, if a cloudy composition is obtained on dilution, the cloudy composition can be left for a number of hours until all components have solubilized and a clear solution is obtained. For example, the cloudy composition may be left for up to 6 hours, up to 12 hours, up to 24 hours or up to 48 hours in order to obtain a clear liquid composition.

To be effective in prevent and/or treating disease in animals, only very low quantities are required to be consumed. Accordingly, the composition of the invention, or indeed the composition for storage/transport may be further diluted before administration to an animal or human in need thereof. Dilution may occur by addition of the composition to a feed, such as a dry feed or liquid feed, or by mixing the composition with drinking water.

To prepare a composition suitable for administration to an animal or human, the composition may be diluted with water.

A composition diluted such that it is suitable for administration may herein be referred to as a ‘composition for administration’. A composition for administration remains a composition according to the invention.

A composition for administration according to the disclosure may comprise up to 96.5%, 97%, 98%, 99% or 99.5% (w/w) water.

‘Dilution’ in the context of the composition for administration may refer to dilution with a liquid or a solid, for example the composition for administration may comprise the composition of the disclosure in combination with a dry feed, or in combination with a liquid.

In an embodiment of the invention, the composition is a liquid composition, the composition for storage/transport is a liquid composition and/or the composition for administration is a liquid composition.

Preferably, liquid compositions according to the disclosure comprise water, preferably wherein the water is drinking water.

When the components are solubilized in the liquid composition, the amount consumed is better regulated, and over-dosing and under-dosing can be avoided.

In particular, providing the composition for administration in drinking water is beneficial as there tends to be less competition for drinking water than for solid food amongst farmed animals. This means that all animals have unrestricted access to the composition for administration, and therefore each animal can consume a sufficient amount to obtain the desired benefits.

Furthermore, animals experiencing symptoms of diarrhoea may avoid solid food and as such, any treatment composition comprised in solid food will not be consumed. Providing the composition of the disclosure in liquid form therefore ensures that animals consume an appropriate amount of the composition in order to prevent sickness and to cure sickness.

In an embodiment, the composition for administration has a low or acidic pH.

A low pH is beneficial for ensuring that all composition components are in solution when the composition comprises water.

In addition, some binding agents require low pH in order to function, or to improve their function. For example, polygalactose is a better binding agent at low pH, for instance at pH below 6. The pH value is of greater importance for some binding agents than others. For example, polygalactose is highly sensitive to the pH whereas tannin components are less sensitive. Accordingly, in embodiments that does not contain polygalactose, the pH may be higher than for embodiments wherein polygalactose is present.

Accordingly, the pH of a liquid composition should be such that all components are solubilized, and have the required functionality. In particular, the pH of the composition for storage/transport and the composition for administration should have a pH suitable for ensuring that all components are solubilized and have the required functionality.

Preferably, the composition has a pH from 1 to 7, such as from 2 to 6, e.g. from 2 to 5 and preferably from 2 to 4. Most preferably, the composition for administration has a pH from 2.5 to 3.6, even more preferably from 2.6 to 3.4.

The composition should comprise an amount of acid component suitable for obtaining a low or acidic pH for the composition for administration, without causing the composition to be so acidic that it is unpalatable to the animal and/or human. The exact amount of acid component required to obtain a pH in the preferred range will depend on the nature of the acid component and the degree of dilution.

Preferably, the composition for administration has a pH from 1 to 7, such as from 2 to 6, e.g. from 2 to 5 and preferably from 2 to 4. Most preferably, the composition for administration has a pH from 2.5 to 3.6, even more preferably from 2.6 to 3.4.

In some embodiments, the composition for administration comprises 0.07-7% (w/w) acid component. The required amount of acid component will depend on the type of acid(s) in the acid component and on the degree of dilution. The amount of acid component in the composition for administration should be such that a pH in the preferred range is obtained.

In some embodiments the range of acid component(s) in the composition for administration is in the range of 0.055% (w/w)-5.5% (w/w), such as 0.080 (w/w)-4 (w/w), e.g. 0.1 (w/w)-3 (w/w), such as 0.2 (w/w)-2 (w/w), e.g. 0.3 (w/w)-1 (w/w), such as 0.4 (w/w)-0.9 (w/w), e.g. 0.5 (w/w)-0.8 (w/w), such as 0.6 (w/w)-0.8 (w/w).

In an embodiment the composition for administration comprises lactic acid component in the range of 0.0054% (w/w)-5.4% (w/w), such as 0.0080 (w/w)-4 (w/w), e.g. 0.01 (w/w)-3 (w/w), such as 0.02 (w/w)-2 (w/w), e.g. 0.03 (w/w)-1 (w/w), such as 0.04 (w/w)-0.9 (w/w), e.g. 0.05 (w/w)-0.8 (w/w), such as 0.06 (w/w)-0.8 (w/w).

In the embodiment the composition for administration comprises lactic acid component in the range of 6.1 10⁻³ M to 6.1 10⁻¹ M such as 7.0 10⁻³ M to 7.0 10⁻¹ M, e.g. 8.0 10⁻³ M to 8.0 10⁻¹ M, such as 1.0 10⁻² M to 8.0 10⁻² M, e.g. 2.0 10⁻² M to 7.0 10⁻² M, such as 3.0 10⁻² M to 7.0 10⁻² M, e.g. 4.0 10⁻² M to 6.0 10⁻² M lactic acid.

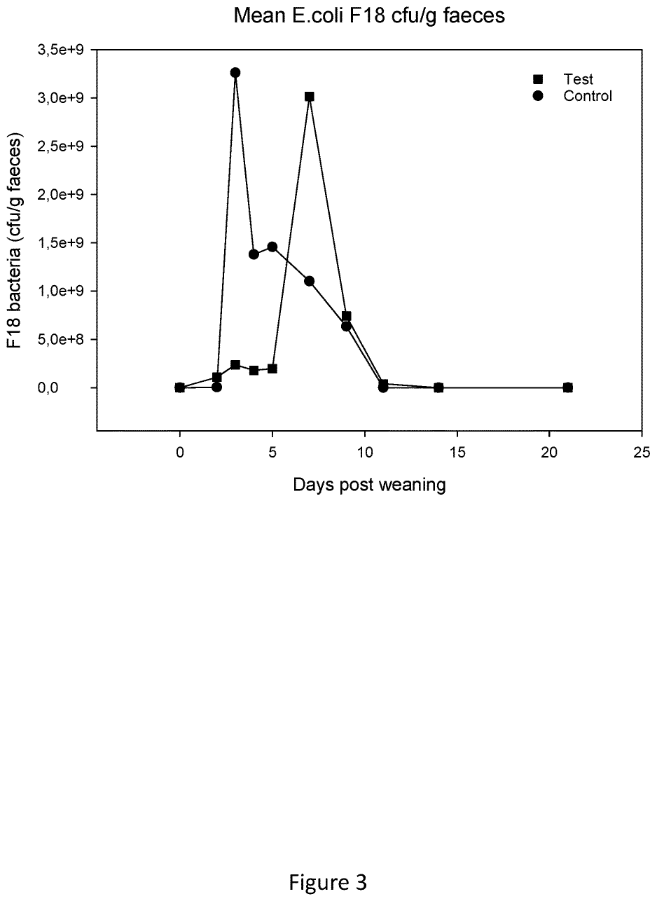

The organoleptic properties (taste, smell, sight and touch) of the composition are extremely important. Piglets will not eat if they dislike the composition and may lose weight, which leaves them vulnerable to infectious diseases. It may therefore be contemplated that compositions comprising lactic acid also comprise a second acid, such as acetic acid, to masks the sour taste of the lactic acid component thus making the composition more attractive for the animals to drink and/or eat. The results of Example 3 (FIG. 3) show that the composition of the invention is indeed highly palatable to piglets.

In an embodiment, the acid component comprises lactic acid and second acid, wherein the second acid is, for example, acetic acid.

In an embodiment, the composition for administration comprises acetic acid as the second acid, in the range of 0.00131% (w/w) to 0.131% (w/w), such as 0.002% (w/w) to 0.09% (w/w), e.g. 0.004% (w/w) to 0.07% (w/w), such as 0.006% (w/w) to 0.05% (w/w), e.g. 0.008% (w/w) to 0.07% (w/w), such as 0.01% (w/w) to 0.09% (w/w).

If the second acid is an acid other than acetic acid, a different amount and/or molar ratio of lactic acid to second acid may be required to obtain a composition for administration with a pH in the preferred range.

In an embodiment the composition for administration comprises a second acid in the range of 2.19 10⁻⁴ M-2.19 10⁻² M, such as 3.00 10⁻⁴ M-1.00 10⁻² M, e.g. 4.00 10⁻⁴ M-9.00 10⁻³ M, such as 5.00 10⁻⁴ M-8.00 10⁻³ M, e.g. 6.00 10⁻⁴ M-7.00 10⁻³ M, such as 7.00 10⁻⁴ M-6.00 10⁻³ M, e.g. 8.00 10⁻⁴ M-5.00 10⁻³ M, such as 9.00 10⁻⁴ M-4.00 10⁻³ M, e.g. 1.00 10⁻³ M-3.00 10⁻³ M and any combination thereof.

In some embodiment, the composition for administration comprises a binding agent in the range of 0.003% (w/w)-1% (w/w), for instance 0.005 (w/w)-0.8 (w/w), 0.006 (w/w)-0.5 (w/w), 0.008 (w/w)-0.2 (w/w), 0.01 (w/w)-0.1% (w/w), 0.02 (w/w)-0.1 (w/w), or 0.025 (w/w)-0. 1 (w/w).

In an embodiment the composition for administration comprises a tannin component in the range of 0.00259 (w/w)-0.9% (w/w), such as 0.003 (w/w)-0.8 (w/w), e.g. 0.005 (w/w)-0.5 (w/w), 0.008 (w/w)-0.7 (w/w), such as 0.01 (w/w)-0.6 (w/w), e.g. 0.01 (w/w)-0.5 (w/w), such as 0.015 (w/w)-0.4 (w/w), e.g. 0.015 (w/w)-0.3 (w/w), such as 0.02 (w/w)-0.2 (w/w), e.g. 0.025 (w/w)-0.1 (w/w).

In an embodiment the composition for administration comprises a tannin component in the range of 1.38 10⁻⁶ M-5.44 10⁻³ M, such as 2.00 10⁻⁶ M-4.00 10⁻³ M, such as 2.00 10⁻⁶ M-1.00 10⁻³ M, e.g. 4.00 10⁻⁶ M-0.50 10⁻³ M e.g. 7.00 10⁻⁶ M-3.00 10⁻³ M, such as 8.00 10⁻⁶ M-2.00 10⁻³ M, e.g. 9.00 10⁻⁶ M-1.00 10⁻³ M, such as 1.00 10⁻⁵M-9.00 10⁻⁴ M, e.g. 1.00 10⁻⁵ M-7.00 10⁻⁴ M and any combination thereof.

In an embodiment the composition for administration comprises a polygalactose component in the range of 0.000259% (w/w)-0.259% (w/w), such as 0.0003 (w/w)-0.2 (w/w), e.g. 0.0005 (w/w)-0.1 (w/w), 0.0008 (w/w)-0.1 (w/w), such as 0.001 (w/w)-0.1 (w/w), e.g. 0.0015 (w/w)-0.1 (w/w), such as 0.005 (w/w)-0.09 (w/w), e.g. 0.002 (w/w)-0.08 (w/w).

The molar mass of the polygalactose component will depend on the size of the polymer. In embodiments wherein the composition comprises a polygalactose component, the composition for administration may comprise a concentration of polygalactose suitable for obtaining a weight ratio of acid component:polygalactose according to the disclosure.

In embodiments wherein the binding agent comprises more than one binding agent component, the weight % of binding agent and/or the amount of each binding agent required in the composition for administration may be significantly lower than for compositions comprising only one type of binding agent. Without wishing to be bound by theory, the combination of the binding agents may provide synergistic effects due to different mechanisms of fimbriae binding accelerating binding rate or increasing the binding efficiency.

For example, in an embodiment the binding agent comprises a tannin component and another binding agent, wherein the composition for administration comprises the tannin component in the range 0.00259% (w/w)-0.259% (w/w), such as 0.003 (w/w)-0.2 (w/w), e.g. 0.005 (w/w)-0.1 (w/w), 0.008 (w/w)-0.1 (w/w), such as 0.01 (w/w)-0.1 (w/w), e.g. 0.08 (w/w)-0.1 (w/w), such as 0.06 (w/w)-0.05 (w/w), e.g. 0.02 (w/w)-0.03 (w/w).

For example, in an embodiment the binding agent comprises a polygalactose component and another binding agent, wherein the composition for administration comprises the polygalactose component in the range of 0.000259% (w/w)-0.0259% (w/w), such as 0.0003 (w/w)-0.02 (w/w), e.g. 0.0005 (w/w)-0.01 (w/w), 0.0008 (w/w)-0.01 (w/w), such as 0.001 (w/w)-0.01 (w/w), e.g. 0.0015 (w/w)-0.01 (w/w), such as 0.005 (w/w)-0.009 (w/w), e.g. 0.002 (w/w)-0.008 (w/w).

Polygalactose is a more effective binding agent that tannin components therefore typically, less polygalactose component is required by weight to achieve the same effect as a tannin component.

For example, when the binding agent is polygalactose, the amount of binding agent required by weight may be up 10×, 7×, 5×, 4× or 2× less than when the binding agent is a tannin component.

In an embodiment the composition for administration comprises 0.7% (w/w) acid component and 0.08-0.1% (w/w) binding agent, wherein the binding agent is a tannin component.

In an embodiment the composition for administration comprises an acid component and 0.08-0.1% (w/w) binding agent, wherein the binding agent is a tannin component, wherein the pH of the composition for administration is from 2.6 to 3.4.

In an embodiment, the composition for administration comprises 0.7% (w/w) acid component and 0.01-0.02% (w/w) binding agent, wherein the binding agent is a polygalactose component.

In an embodiment the composition for administration comprises an acid component and 0.01-0.02% (w/w) binding agent, wherein the binding agent is a polygalactose component, wherein the pH of the composition for administration is from 2.6 to 3.4.

In a preferred embodiment the composition for administration comprises 0.7% acid component and 0.02-0.03% (w/w) binding agent, wherein the binding agent comprises a tannin component and a polygalactose component.

In a preferred embodiment the composition for administration comprises 0.02-0.03% (w/w) binding agent, wherein the binding agent comprises a tannin component and a polygalactose component, wherein the pH of the composition for administration is from 2.6 to 3.4

In a preferred embodiment the composition for administration comprises 0.7% (w/w) acid component(s) or a salt or ester thereof, 0.0259% (w/w) tannin component or a salt or ester thereof, and 0.00259% (w/w) polygalactose component.

In a preferred embodiment the composition for administration comprises an acid component(s) or a salt or ester thereof, 0.0259% (w/w) tannin component or a salt or ester thereof, and 0.00259% (w/w) polygalactose component, wherein the pH of the composition is from 2.6 to 3.4.

In a preferred embodiment the composition for administration comprises 0.69% (w/w) lactic acid component or a salt or ester thereof, 0.014% (w/w) acetic acid component or a salt or ester thereof, 0.0259% (w/w) tannin component or a salt or ester thereof, and 0.00259% (w/w) polygalactose component.

In a preferred embodiment the composition for administration comprises lactic acid component or a salt or ester thereof, an acetic acid component or a salt or ester thereof, 0.0259% (w/w) tannin component or a salt or ester thereof, and 0.00259% (w/w) polygalactose component, wherein the pH of the composition for administration is 2.6 to 3.4

Without being bound by theory the composition of the invention stimulates the piglet's ability to absorb nutrients from the feed while at the same time making the intestinal wall increasingly more resistant to pathogenic bacteria.

The amount of lactic acid component can be quantified in water using, for instance, a lactate meter like “lactate Pro 2” from Praxisdienst. The amount of acetic acid component and such as acetic acid or a salt or ester thereof can be quantified in water by using, for instance, the acetic acid assay kit from Megazyme. The content of tannic acid or a salt or ester thereof can be quantified, for instance, by the tannic acid/lignin test from Hach. The polygalactose content of the composition can be quantified, for instance, by ¹H-NMR E. Tojo and J. Prado, 2003, Carbohydrate polymers 53, 325-32.

The composition of the invention may be a feed, a food, or a food additive, or may be comprise in a feed, food or food additive. The composition may be present in the food, feed or food additive as the composition or as the composition for administration.

A “feed” and a “food,” respectively, means any natural or artificial diet, meal or the like or components of such meals intended or suitable for being eaten, taken in, digested, by an animal and a human being, respectively.

As used herein, the term “food” is used in a broad sense—and covers food and food products for humans as well as food for animals (i.e. a feed). The term “foodstuff” encompasses any ingredient which may be used for food.

The term “feed” is used with reference to products that are fed to animals in the rearing of livestock. The term “feed” may encompass a feed per se or a feed composition or a component thereof. The terms “feed” and “animal feed” are used interchangeably. In a preferred embodiment, the food or feed is for consumption by an animal. The term “feedstuff” encompasses any ingredient which may be used for feed.

The food or feed may be in the form of a liquid (e.g. a solution) or as a solid—depending on the use and/or the mode of application and/or the mode of administration.

As used herein the term “food or feed ingredient” includes a formulation, which is or can be added to foods or foodstuffs and includes formulations which can be used at low levels in a wide variety of products. The food ingredient may be in the form of a liquid (e.g. a solution) or as a solid depending on the use and/or the mode of application and/or the mode of administration. The composition of the present invention may be added to—food supplements.

In one embodiment, the food or animal feed is a liquid such as a liquid feed. In such embodiments, it is preferable that the acid component and binding agent are solubilized in the liquid feed.

In another embodiment, the food or animal feed is a solid.

In one embodiment, the animal feed is feed for pigs (pig feed) and in particular piglets.

The food or animal feed may comprise vegetable proteins. Vegetable proteins may be derived from legumes, oil seeds, nuts and cereals. Examples of sources of vegetable proteins.

Suitable sources of vegetable proteins are soy beans, soy bean meal, cereals (such as maize (corn), wheat, oats, barley, rye, and sorghum), cereal meals (such as corn meal, wheat meal, oat meal, barley meal, rye meal, sorghum meal, and canola meal), brans (such as what bran, and oat bran), oil seeds (such as rapeseed and sunflower seeds), oil seed meals (such as rapeseed meal), cottonseed meal, cabbage, beet and sugar beet. These vegetable proteins are examples of food ingredients and animal feed ingredients.

The food or animal feed may comprise animal proteins. Suitable animal proteins include, but are not limited to, fish meal and whey. These animal proteins are examples of food ingredients and animal feed ingredients.

The food or animal feed may comprise additives. Suitable additives include, but are not limited to, enzyme inhibitors, vitamins, trace minerals, minerals, colouring agents, aroma compounds and antimicrobial peptides. These additives are examples of food ingredients and animal feed ingredients.

The food or animal feed or ingredients thereof may be a liquid.

As used herein, the term “digestibility” refers to the ability of an animal to absorb or retain nutrition from a food or feed rather than excreting it. The word “nutrition” in this context may include minerals, fats, lipids, vitamins, proteins, amino acids, carbohydrates, and starches. A measure of digestibility is the amount of any aspect of nutrition which is retained by the animal rather than excreted. This may be expressed as a percentage.

The composition of the invention is further suitable for feeding an animal selected from the group consisting of a monogastric farm animal; a monogastric animal; a bird; a poultry bird; a chicken; a duck; a turkey; a pig; a piglet; a weaning piglet; a post-weaning piglets; a swine; a hog; a grower-finisher; a sow; a non-monogastric animal; a ruminant animal; a human, a non-monogastric farm animal; a beef producing animal; a dairy producing animal; an Alpaca; a Bison; a Bovine animal; a Camel; an animal of the Cattle family; a Cow; a Deer; a Donkey; an Equus animal; a Goat; a Horse; a Lamb; any animal considered to be Livestock; a Llama; a Mule; an Ox; a Reindeer; a Sheep; a Steer; a Yak; a Buffalo; a Giraffe; a Moose; a Elk; a Llama; an Antelope; a Pronghorn; a Nilgai; an equine, bovine, cervidae, caprinae, camelidae animal; a fish; a gastric fish; an agastric fish; a shrimp or other crustacean; a marine fish, freshwater fish and any combination thereof. Preferably, the animal is a human or a domesticated animal. Even more preferably the animal is selected from the group consisting of a pig; a piglet; a weaning piglet; a post-weaning piglet; a swine; a hog; a grower-finisher and/or a sow.

Zinc is often used in piglet feed to prevent and/or treat the symptoms of diarrhoea, for example, post-weaning diarrhoea. The composition of the invention is also able to prevent and/or treat the symptoms of diarrhoea and as such, the composition of the invention may be free from medical zinc as shown in Example 2.

A further embodiment of the invention relates to a feed additive, a feed and/or a pre-mix comprising the composition of the invention. In an embodiment the feed additive, the feed and/or the pre-mix comprises at least 0.05% w/w of the composition of the present invention, preferably at least 0.05-3%, such as 0.1-6%, e.g. 0.1-9%, more preferably 0.5-10% and most preferably 15% w/w. The disclosure also relates to a feed comprising the composition according to the present invention and at least one mineral and/or at least one vitamin. In an embodiment the feed composition comprises at least 0.05% w/w of the composition of the present invention, preferably at least 0.05-3%, such as 0.1-6%, e.g. 0.1-9%, more preferably 0.5-10% and most preferably 15% w/w.

Other embodiments of the invention include a feed additive comprising the composition the invention and at least a mineral and/or at least one vitamin, a pre-mix comprising the composition of the present invention and at least a mineral and/or at least one vitamin. The disclosure also relates to a kit comprising the composition of the invention, the feed of the invention, the feed additive of the invention, and/or the pre-mix of the invention and instructions for administration.

The early weeks after weaning are critical to a piglets development, and it is of utmost importance that weight is gained and sickness is avoided in these weeks. The disclosure also relates to use of the composition of the invention for feeding weaning piglets for 0-5 weeks post-weaning. In an embodiment the period is 1-4 weeks, such as 2-3 weeks and any combinations thereof.

Biophysical characteristics can be used as an indicator of the development of animals, such as weaning piglets and as such, the disclosure also relates to the use of the composition according the present invention, for improving or maintaining the biophysical characteristics of an animal (such as a weaning piglet) compared to an animal (such as a weaning piglet) that have not received the composition of the invention.

An animal's biophysical characteristic “as a food source” refers to biophysical characteristics which increase the value of the animal as a food source, particularly a human food source. These biophysical characteristics may improve the health, weight, size, fat content, rate of growth, time to reach maturity, taste and other characteristics related to ease of use in food production. In addition, these biophysical characteristics may include but are not limited to the following: maturity and health of an animal. Such an animal with these improved biophysical characteristics may have higher value as food, a higher market value, better taste, more nutritional value and be used in different cooking methods. A “food source” may encompass any aspect of an animal such as meat, protein, fat, coat or fur, feathers, milk, or eggs.

In an embodiment, the biophysical characteristics are selected from the group consisting of;
a) An increase in feed conversion ratio (FCR)
b) An increase in weight
c) An increase in drinking water consumption
d) An increase in the dry matter content of faeces,
e) A decrease in symptoms of PWD, and/or
f) An increase in feed consumption

As used herein, “improving” refers to an improvement compared to the equivalent use of a composition not comprising acid component(s), and a binding agent. Such an improvement may comprise an increase or decrease in a characteristic. The improvement in one or more biophysical characteristics may be at least 0.5%, at least 1%, at least 2%, at least 3%, at least 5%, at least 10%, at least 12%, at least 15%, at least 20% or at least 30%. In some embodiments the improvement may be at least 50% or at least 100% or at least 150%.

The percentage improvement in biophysical characteristics may be in one aspect in comparison to the use of a control composition not comprising acid component(s), a binding agent component.

The increase in FCR of an animal may be at least 0.5%, at least 1%, at least 2%, at least 3%, at least 5%, at least 10%, at least 12%, at least 15%, at least 20% or at least 30%. In some embodiments the increase may be at least 50% or at least 100%. The increase in FCR of an animal may be in respect to a control composition in which the composition used does not comprise acid component(s) in combination with a binding agent.

The increase in weight gain of an animal may be at least 0.5%, at least 1%, at least 2%, at least 3%, at least 5%, at least 10%, at least 12%, at least 15%, at least 20% or at least 30% per week. In some embodiments the increase may be at least 50% or at least 100% per week. In another embodiment the increase in weight of an animal is 10-20% per week. In a specific embodiment the increase in weight of an animal is in the range of 0.5 and 1.3 kg a week, for example 0.8 kg per animal per week. The increase in weight gain of an animal may be in respect to a control composition in which the composition used does not comprise acid component(s) in combination with a binding agent.

The increase in drinking water consumption by an animal may be at least 0.5%, at least 1%, at least 2%, at least 3%, at least 5%, at least 10%, at least 12%, at least 15%, at least 20% or at least 30%. In some embodiments the increase may be at least 50% or at least 100%. The increase in drinking water consumption by an animal may be in respect to a control composition in which the composition used does not comprise acid component(s) in combination with a binding agent.

The increase in the dry matter content of faeces of an animal may be at least 0.5%, at least 1%, at least 2%, at least 3%, at least 5%, at least 10%, at least 12%, at least 15%, at least 20% or at least 30%. In some embodiments the increase may be at least 50% or at least 100%. The increase in dry matter content of faeces of an animal may be in respect to a control composition in which the composition used does not comprise acid component(s) in combination with a binding agent.

The increase in feed consumption of an animal may be at least 0.5%, at least 1%, at least 2%, at least 3%, at least 5%, at least 10%, at least 12%, at least 15%, at least 20% or at least 30%. In some embodiments the increase may be at least 50% or at least 100%. The increase in feed consumption of an animal may be in respect to a control composition in which the composition used does not comprise acid component(s) in combination with a binding agent.

The decrease in symptoms of PWD of an animal and in particular a piglet may be at least 0.5%, at least 1%, at least 2%, at least 3%, at least 5%, at least 10%, at least 12%, at least 15%, at least 20% or at least 30%. In some embodiments the decrease may be at least 50% or at least 100%. The decrease in symptoms of PWD of an animal and in particular a piglet may be in respect to a control composition in which the composition used does not comprise acid component(s) in combination with a binding agent, and the decrease may be in relation to any of the symptoms related to PWD.

In an embodiment the invention relates to the use of the composition of the present invention in combination with a feed free from medical zinc. An important embodiment relates to the use of the composition of the present invention, the feed of the present invention, the feed additive according to of the present invention or the pre-mix according to of the present invention for reducing the use of antibiotics and/or medical zinc oxide while increasing or maintaining the biophysical characteristic of weaning piglets.

In a specific embodiment the invention relates to the use of the composition of the invention for increasing weight gain in weaning piglets relative to weaning piglets that did not consume the composition or which weaning piglets received a control composition not comprising acid component(s) in combination with a binding agent but otherwise identical to the composition of the present invention.

Likewise in another embodiment the invention relates to the use of the composition of the invention for increasing the dry matter content in faeces of piglets with post-weaning diarrhoea compared to weaning piglets with PWD that did not consume the composition of the invention or which weaning piglets received a control composition not comprising acid component(s) in combination with a binding agent but otherwise identical to the composition of the present invention.

In a relevant embodiment the invention relates to the use of the composition of the invention for maintaining, re-establishing, or increasing the dry matter content in faeces of piglets with post-weaning diarrhoea. Time is key when raising piglets and the dry matter content in faeces may be indicative or a symptom of PWD. Not all piglets show symptoms of PWD and for this group it's important to maintain the dry matter content in faeces. For the group of piglets showing symptoms of PWD it is important to either re-establish or even increase the dry matter content in faeces. The invention therefore also relates to the use of the composition of the invention for preventing growth of E-coli in the gastrointestinal tract of an animal. In an equally relevant embodiment, the invention is also directed to the use of the composition of the invention to decrease the number of pathogenic bacteria in the gastrointestinal tract of an animal.

In another embodiment the invention relates to the composition of the present invention for use in medicine. More specifically the invention relates to the composition of the invention for use in preventing and/or treating post-weaning diarrhoea in animals and preferably in a piglet. The inventors discovered that the use of the composition of the invention improved resistance to entero toxigenetic E coli bacteria in weaning piglets (see Example 3). Thus, in one embodiment the invention relates to the composition of the invention for use in improving resistance to entero toxigenetic E coli bacteria in an animal that did not consume the composition of the invention or which animal received a control composition not comprising acid component(s) in combination with a binding agent but otherwise identical to the composition of the present invention.

In a specific embodiment the invention relates to the composition of the invention for use in improving resistance to entero toxigenetic E. coli bacteria in a weaning piglet compared to a weaning piglet that did not consume the composition of the invention or which weaning piglet received a control composition not comprising acid component(s) in combination with a binding agent but otherwise identical to the composition of the present invention. The disclosure therefore is also directed to a method of preventing and/or treating PWD in a piglet by administering the composition of the invention.

The larger the animal, the more of the composition and/or binding agent required to obtain the desired effect. Typically, the larger the animal the more food and/or drinking water consumed each day.

Accordingly, when the composition is administered in food/drinking water, the food/drinking water should comprise sufficient binding agent to be effective. Typically, the amount of food/drinking water consumed increase as an animal's weight increase.

In the context of the disclosure, an effective amount of binding agent may be considered at least 0.01 g binding agent per kg body weight per day, for example 0.1 g, 0.2 g or 0.3 g binding agent per kg body weight per day. For effective binding agents such as polygalactose, a lower amount may need to be consumed. Accordingly, an effective amount of binding agent may also be considered the amount required to obtain the desired effect of preventing/treating an animal in need thereof.

An embodiment of the invention therefore relates to use of a binding agent for preventing and/or treating sickness in an animal in need thereof, wherein the animal consumes at least 0.01 g binding agent per kg body weight per day, for example 0.1 g, 0.2 g or 0.3 g binding agent per kg body weight per day.

Typically, piglets consume between 1.5 L and 3 L of drinking water a day for 0-5 days after weaning, and up to 5-6 L per day 12-21 days after weaning.

A further embodiment of invention relates to use of a binding agent for preventing and/or treating sickness in an animal in need thereof, wherein the animal consumes at least 0.01 g binding agent per kg body weight per day, for example 0.1 g, 0.2 g or 0.3 g binding agent per kg body weight per day by consuming between 1.5 L and 5 L of the composition for administration per day, wherein the composition for administration is administered via drinking water.

In an embodiment, the invention relates to a method of preparing the composition of the invention, said method comprising the steps of:
a) Combining an acid component and a binding agent.

In an embodiment, the invention relates to a method of preparing the composition of the invention, said method comprising the steps of:
a) Combining an acid component and a binding agent; and
b) a liquid, wherein
the pH of the composition is between pH from 1 to 7, such as from 2 to 6, e.g. from 2 to 5 and preferably from 2 to 4, most preferably, from 2.5 to 3.6, even more preferably from 2.6 to 3.4.

In an embodiment, the invention relates to a method of preparing the composition of the invention, said method comprising the steps of:
a) Combining an acid component and a binding agent, wherein, the weight ratio of acid component to binding agent is 1-150:1-20; for instance, 1-100:1-20; 1-50:1-20; 5-50:1-20; 5-50:1-10; 5-50:1-5; 5-50:1-2. Preferably the weight ratio of acid component to binding agent is approximately 5-50:1.

Another embodiment the invention relates to a method of preparing the composition, said method comprising the steps of:
a) Combining a lactic acid component, a second acid component and a binding agent;
   wherein the binding agent comprises a tannin component, wherein the tannin component is preferably tannic acid, and/or a polygalactose component.

In an alternative embodiment the invention is directed a method of preparing the composition of the invention, said method comprising the steps of:
a) providing a feed comprising a protein source, a carbohydrate source, a lipid source and optionally one or more ingredients selected from the group consisting of fish meal, vitamins, mineral premix and combinations thereof,
b) providing an acid component(s) and a binding agent;
wherein the binding agent comprises tannic acid and/or polygalactose; and
c) obtaining a composition comprising
an acid component(s),
a binding agent comprising a tannin component, and/or a polygalactose component.

In a further embodiment of the invention relates to a method of administering the composition of the invention to an animal in need thereof, said method comprising the steps of:
a) Combining the composition of the invention with drinking water,
b) Obtaining a composition for administration,
c) and providing the animal in need thereof with the composition for administration of step b).

In a further embodiment of the invention relates to a method of preparing the composition, said method comprising the steps of:
a) Combining the composition of the invention with drinking water,
b) Obtaining a composition for storage and/or transport.

In a further embodiment of the invention relates to a method of preparing the composition, said method comprising the steps of:
a) Combining the composition of the invention with drinking water,
b) Obtaining a composition for administration,
c) and providing the animal in need thereof with the composition for administration of step b).

In an embodiment the drinking water comprising the composition of the invention is administered via a dosing chamber in the form of a composition for administration, wherein the drinking water comprises an amount of acid component(s) between 0.055%-5.5% w/w combined with a binding agent in an amount between 0.003% (w/w)-1% (w/w), preferably wherein the binding agent comprises a tannin component and/or a polygalactose component, wherein, when present, the tannin component is in an amount between 0.00259 (w/w)-0.9% (w/w), w/w and, when present, the polygalactose component is in an amount between 0.000259% (w/w)-0.259%.

In an embodiment the drinking water comprising the composition of the invention is administered via a dosing chamber in the form of a composition for administration wherein the pH of the composition for administration is from 1 to 7, such as from 2 to 6, e.g. from 2 to 5 and preferably from 2 to 4, most preferably, the composition for administration has a pH from 2.5 to 3.6, even more preferably from 2.6 to 3.4.

In a relevant embodiment the present invention relates to a method of administering the composition for administration in the form of a liquid composition, the method comprising; pouring the liquid composition over a dry feed, wherein the liquid composition is poured over the dry feed at least twice every 24 hours, wherein at least 0.2 L of the liquid composition is provided per piglet twice a day, and wherein the dry feed is free from medical zinc.

The disclosure further relates to a method of feeding an animal comprising administering to said animal the composition according to the invention.

The method(s) of feeding described herein may involve any steps which can normally be used in feeding animals. In one aspect the method of feeding an animal with a feed is not a method of treatment or therapy.

It will be appreciated that aspects and embodiments disclosed in the parts related to the composition, or the composition for storage/transport, or the composition for administration and the method of preparing the composition, or the composition for administration may be equally relevant to all other parts of the specification.

The use of the terms “a” and “an” and “the” and similar referents in the context of describing the invention (especially in the context of the claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The listing or discussion of an apparently prior published document in this specification should not necessarily be taken as an acknowledgement that the document is part of the state of the art or is common general knowledge.

Preferences, options and embodiments for a given aspect, feature or parameter of the invention should, unless the context indicates otherwise, be regarded as having been disclosed in combination with any and all preferences, options and embodiments for all other aspects, features and parameters of the invention. Embodiments and features of the present invention are also outlined in the items.

### Items

Item A1. A composition for an animal comprising an acid component and a binding agent.

Item A2. The composition of item A1, wherein the weight ratio of acid component to binding agent is 1-150:1-20; for instance, 1-100:1-20; 1-50:1-20; 5-50:1-20; 5-50:1-10; 5-50:1-5; 5-50:1-2. Preferably the weight ratio of acid component to binding agent is approximately 5-50:1.

Item A3. The composition of any items A1-A2, comprising 70-99.9% (w/w), such as 72-99.5% (w/w), 75-99% (w/w), 80-99% (w/w) or 82-98% (w/w) acid component; and 0.1-30% (w/w), such as 0.5-28% (w/w), 1-25% (w/w), 1-20% (w/w), or 2-18% (w/w) binding agent.

Item A4. The composition of items A1-A3 wherein the composition is a liquid composition.

Item A5. The composition of any of items A1-A4 wherein the composition comprises a liquid.

Item A6. The composition of item A5 wherein the liquid is water, preferably wherein the liquid is drinking water.

Item A7. The composition of any of items A3-A6 wherein the binding agent and the acid component are in solution.

Item A8. The composition of any of items A1-A7 wherein the composition is acidic.

Item A9. The composition of any one of items A3-A8, wherein the pH is in the range of 1 and 7, such as from 2 to 6, e.g. from 2 to 5 and preferably from 2 to 4, most preferably, from 2.5 to 3.6, even more preferably from 2.6 to 3.4.

Item A10. The composition of any of items A1-A9 wherein the acid component comprises one, two or more acids.

Item A11. The composition of item any preceding claim, wherein the acid component is selected from acetic acid, hydrochloric acid, phosphoric acid, nitric acid, citric acid or sulfuric acid, or combinations thereof, preferably wherein the second acid is acetic acid.

Item A12. The composition of any of items A1-A11 wherein the composition comprises lactic acid.

Item A13. The composition of any of items A1-A12 wherein the composition comprises a lactic acid and a second acid.

Item A14. The composition of item A13, wherein the second acid is selected from acetic acid, hydrochloric acid, phosphoric acid, nitric acid, citric acid or sulfuric acid, or combinations thereof, preferably wherein the second acid is acetic acid.

Item A15. The composition of any of items A13-A14 wherein the weight ratio of second acid to lactic acid is in the range 1-5:20-150, such as 1-5:25-100, e.g. 1-5:25-75, such as 1-2:25-60.

Item A16. The composition of any of items A13-A15 wherein the amount of second acid is sufficient to improve the organoleptic properties of the composition.

Item A17. The composition of any of items A1-A16 wherein the binding agent is an E. coli binding agent.

Item A18. The composition of any of items A1-A17 wherein the binding agent comprises a plurality of E. coli binding sites.

Item A19. The composition of item A1-A18 wherein the binding agent comprises a tannin component.

Item A20. The composition of item A19 wherein the tannin component is tannic acid, or a salt or ester thereof.

Item A21. The composition of item A19-20, wherein the tannin component comprises a plurality of E.coli binding sites.

Item A22. The composition of item A19-21, wherein the tannin component can bind E. coli at two, three, four, five or more sites simultaneously.

Item A23. The composition of any of items A19-A22 wherein the tannin component can bind the lengths of E. coli fimbriae.

Item A24. The composition of any of items A19-A23, wherein the weight ratio of acid component:tannin component is in the range 2-200:1, preferably the weight ratio is 2-100:1, for instance 2-50:1, 5-40:1, or 5-30:1.

Item A25. The composition of any of items A1-A24 wherein the binding agent comprises a polygalactose component.

Item A26. The composition of item A25 wherein the polygalactose component is water-soluble.

Item A27. The composition of item A22 wherein water-soluble is defined as a compound that produces a clear or transparent liquid without precipitate when 1 g is added to 1 L of water at 25° C.

Item A28. The composition of item A25-27 wherein the polygalactose component is a sulphated polygalactose component.

Item A29. The composition of any of items A25-A28 wherein the polygalactose component is a carrageenan, preferably wherein the polygalactose component is kappa-carrageenan.

Item A30. The composition of any of item A25-A29 wherein the weight ratio of acid component:polygalactose component is in the range 5-350:1, preferably the weight ratio is 20-300:1, for instance 50-300:1, 100-300:1, or 200-300:1.

Item A31. The composition of any of items A25-A30 wherein the polygalactose components comprises a plurality of E. coli binding sites.

Item A32. The composition of any of items A25-A31 wherein the polygalactose component can bind E.coli at two, three, four, five or more sites simultaneously.

Item A33. The composition of any of items A25-A32 wherein the polygalactose component can bind the tips of E.coli fimbriae.

Item A34. The composition of any of items A25-A33 wherein the polygalactose component can bind the lengths of E.coli fimbriae.

Item A35. The composition of any of items A25-A34 wherein the polygalactose component can bind the length and the tips of E.coli fimbriae, preferably simultaneously.

Item A36. The composition of any of items A25-A35 wherein the polygalactose component is a polysaccharide.

Item A37. The composition of any of items A25-AA36 wherein the polygalactose component comprises 16 or more monosaccharide sub-units.

Item A38. The composition of any of items A25-A37 wherein the polygalactose component has a weight average molecular weight between 10,000 Da and 1,000,000 Da, such as between 50,000 Da and 75,000 Da, preferably the weight average molecular weight is between 150,000 Da and 300,000 Da as determined via size exclusion chromatography coupled with a multi-angled laser light scattering.

Item A39. The composition of any of items A1-A38 wherein the binding agent comprises a tannin component and a polygalactose component.

Item A40. The composition of item A39, wherein the weight ratio of acid component:tannin component:polygalactose component is in the range of acid component(s):tannin component:polygalactose component is in the range of 100-300:5-20:1, for instance 200-300:5-15:1, 250-300:8-12:1. Preferably, the weight ratio of acid component(s):tannin component:polygalactose component is approximately 269:9.5:1.

Item A41. The composition according to any one of items A1-A40, wherein the composition is free from medical zinc.

Item A42. A composition for storage/transport comprising the composition of any preceding item, wherein the composition for storage and/or transport comprises:
an acid component;
a binding agent; and
up to 20%, 30%, 40% or 50% (w/w) water.

Item A43. The composition for administration of any one of items A44-A56, wherein the acid component is included in an amount sufficient to produce a composition for administration with a pH of from 1 to 7, such as from 2 to 6, e.g. from 2 to 5 and preferably from 2 to 4, most preferably from 2.5 to 3.6, even more preferably from 2.6 to 3.4.

Item A44. A composition for administration comprising the composition of any preceding item, wherein the composition for administration comprises;
An acid component
A binding agent; and
up to 96.5%, 97%, 98%, 99% or 99.5% (w/w) water.

Item A45. The composition for administration of item A44 wherein the pH is in the range of 1 and 7, for instance 2 and 6, 2 and 5 and preferably 2 and 4, most preferably between 2.5 and 3.5, even more preferably from 2.6 to 3.4.

Item A46. The composition for administration of items A44-A45 wherein the acid component(s) is in the range of 0.055% (w/w)-5.5% (w/w), such as 0.080 (w/w)-4 (w/w), e.g. 0.1 (w/w)-3 (w/w), such as 0.2 (w/w)-2 (w/w), e.g. 0.3 (w/w)-1 (w/w), such as 0.4 (w/w)-0.9 (w/w), e.g. 0.5 (w/w)-0.8 (w/w), such as 0.6 (w/w)-0.8 (w/w).

Item A47. The composition for administration of any of items A44-A46 wherein lactic acid is in the range in the range of 0.0055% (w/w)-5.5% (w/w), such as 0.0080 (w/w)-4 (w/w), e.g. 0.01 (w/w)-3 (w/w), such as 0.02 (w/w)-2 (w/w), e.g. 0.03 (w/w)-1 (w/w), such as 0.04 (w/w)-0.9 (w/w), e.g. 0.05 (w/w)-0.8 (w/w), such as 0.06 (w/w)-0.8 (w/w).

Item A48. The composition for administration of any of items A44-A47 comprising a second acid, wherein the second acid is acetic acid, and is included in an amount in the range of 0.0014% (w/w) to 0.14% (w/w), such as 0.002% (w/w) to 0.1% (w/w), e.g. 0.004% (w/w) to 0.08% (w/w), such as 0.006% (w/w) to 0.07% (w/w), e.g. 0.008% (w/w) to 0.06% (w/w), such as 0.01% (w/w) to 0.03% (w/w), preferably 0.01% (w/w) to 0.015 wt %.

Item A49. The composition for administration of any of items A44 to A48 wherein the amount of binding agent is in the range 0.003% (w/w)-0.9% (w/w), for instance 0.005 (w/w)-0.8 (w/w), 0.006 (w/w)-0.5 (w/w), 0.008 (w/w)-0.2 (w/w), 0.01 (w/w)-0.1 (w/w), 0.02 (w/w)-0.1 (w/w), or 0.025 (w/w)-0.1 (w/w).

Item A50. The composition for administration of any of items A44-A49 wherein the amount of tannin component is in the range 0.00259 (w/w)-0.9% (w/w), such as 0.003 (w/w)-0.8 (w/w), e.g. 0.005 (w/w)-0.5 (w/w), 0.008 (w/w)-0.7 (w/w), such as 0.01 (w/w)-0.6 (w/w), e.g. 0.01 (w/w)-0.5 (w/w), such as 0.015 (w/w)-0.4 (w/w), e.g. 0.015 (w/w)-0.3 (w/w), such as 0.02 (w/w)-0.2 (w/w), e.g. 0.025 (w/w)-0.1 (w/w).

Item A51. The composition for administration of any of items A44-A50 wherein the amount of polygalactose component is in the range 0.000259% (w/w)-0.259% (w/w), such as 0.0003 (w/w)-0.2 (w/w), e.g. 0.0005 (w/w)-0.1 (w/w), 0.0008 (w/w)-0.1 (w/w), such as 0.001 (w/w)-0.1 (w/w), e.g. 0.0015 (w/w)-0.1 (w/w), such as 0.005 (w/w)-0.09 (w/w), e.g. 0.002 (w/w)-0.08 (w/w). Item A52. The composition for administration of any of items A44-A52 wherein the binding agent comprises a tannin component and another binding agent, wherein the tannin component in the range 0.00259% (w/w)-0.259% (w/w), such as 0.003 (w/w)-0.2 (w/w), e.g. 0.005 (w/w)-0.1 (w/w), 0.008 (w/w)-0.1 (w/w), such as 0.01 (w/w)-0.1 (w/w), e.g. 0.08 (w/w)-0.1 (w/w), such as 0.06 (w/w)-0.05 (w/w), e.g. 0.02 (w/w)-0.03 (w/w).

Item A53. The composition for administration of any of items A44-A52 wherein the binding agent comprises a polygalactose component and another binding agent, wherein the polygalactose component in the range of 0.000259% (w/w)-0.0259% (w/w), such as 0.0003 (w/w)-0.02 (w/w), e.g. 0.0005 (w/w)-0.01 (w/w), 0.0008 (w/w)-0.01 (w/w), such as 0.001 (w/w)-0.01 (w/w), e.g. 0.0015 (w/w)-0.01 (w/w), such as 0.005 (w/w)-0.009 (w/w), e.g. 0.002 (w/w)-0.008 (w/w).

Item A54. The composition for administration of any of items A44-A53, comprising an acid component and 0.08-0.1% (w/w) binding agent, wherein the binding agent is a tannin component, wherein the pH of the composition for administration is from 2.6 to 3.4.

Item A55. The composition for administration of any of items A44-A42 comprising 0.01-0.02% (w/w) binding agent, wherein the binding agent is a polygalactose component, wherein the pH of the composition for administration is from 2.6 to 3.4.

Item A56. The composition for administration for administration of any of items A44-A55 comprising 0.02-0.03% (w/w) binding agent, wherein the binding agent comprises a tannin component and a polygalactose component, wherein the pH of the composition for administration is from 2.6 to 3.4

Item A57. A feed, a feed additive and/or a pre-mix comprising the composition according to any one of items A1-A43 or the composition for administration of A44-A56.

Item A58. The feed, feed additive and/or premix according to item A57 wherein the feed comprises at least 0.05% w/w of the composition of any one of items A1-A43 or the composition for administration of A44-A56.

Item A59. A pre-mix comprising the composition according to any one of items A1-A43 or the composition for administration of A44-A56 and at least one mineral and/or at least one vitamin.

Item A60. A kit comprising the composition according to items A1-A43 the composition for administration according to any of items A44-A56, the feed, feed additive and/or pre-mix according to items A57-A59, and instructions for administration.

Item A61. The composition according to any one of the items A1-A43, the composition for administration of any one of items A44-A56, the feed, feed additive and/or premix of items A57-A59 wherein the animal is selected from a list comprising a monogastric farm animal; a monogastric animal; a bird; a poultry bird; a chicken; a duck; a turkey; a pig; a piglet; a weaning piglet; a post-weaning piglet; a swine; a hog; a grower-finisher; a sow; a non-monogastric animal; a ruminant animal; a non-monogastric farm animal; a beef producing animal; a dairy producing animal; an Alpaca; a Bison; a Bovine animal; a Camel; an animal of the Cattle family; a Cow; a Deer; a Donkey; an Equus animal; a Goat; a Horse; a Lamb; any animal considered to be Livestock; a Llama; a Mule; an Ox; a Reindeer; a Sheep; a Steer; a Yak; a Buffalo; a Giraffe; a Moose; a Elk; a Llama; an Antelope; a Pronghorn; a Nilgai; an equine, bovine, cervidae, caprinae, camelidae animal; a fish; a gastric fish; an agastric fish; a shrimp or other crustacean; a marine fish or a freshwater fish and a human, preferably wherein said animal is a human or a domesticated animal, preferably wherein the animal is selected from a pig; a piglet; a weaning piglet; a post-weaning piglets; a swine; a hog; a grower-finisher or a sow

Item A62. The feed, feed additive and/or premix according to any of items A57-A59 wherein the composition of any of items A1-A43 is present in the form of the composition for administration of any one of items A44-A56.

Item B1. Use of a tannin compound, a polygalactose compound, or a combination thereof for improving or maintaining the biophysical characteristics of animals compared to animals that do not receive the composition, and wherein the biophysical characteristic is at least one characteristic selected from the group consisting of:
a) An increase in feed conversion ratio
b) An increase in weight
c) An increase in drinking water consumption
d) An increase in the dry matter content of faeces,
e) A decrease in symptoms of PWD
f) An increase in feed consumption.

Item B2. Use of the composition according to any one of items A1-A43, or the composition for administration of any one of items A44-A56, or the feed, feed additive or premix according to any of items A57-59 for improving or maintaining the biophysical characteristics of animals compared to animals that do not receive the composition, and wherein the biophysical characteristic is at least one characteristic selected from the group consisting of:
a) An increase in feed conversion ratio
b) An increase in weight
c) An increase in drinking water consumption
d) An increase in the dry matter content of faeces,
e) A decrease in symptoms of PWD
f) An increase in feed consumption.

Item B3. Use of the composition according to any one of items A1-A43 or the composition for administration of any one of items A44-A56, or the feed, feed additive or premix according to any of items A57-59 in combination with a feed free from medical zinc.

Item B4. Use of the composition according to items A1-A43, the composition for administration of any one of items A44-A56 the feed, feed additive and/or premix of any of items A57-A59 for reducing the use of antibiotics and/or medical zinc oxide while increasing or maintaining the biophysical characteristic of weaning piglets.

Item B5. Use of the composition according to any one of claims items A1-A43 or the composition for administration of any one of items A44-A56 or the feed, feed additive or premix according to any of items A57-59 for increasing weight gain in weaning piglets relative to weaning piglets who did not consume the composition.

Item B6. Use of the composition according to any one of claims items A1-A43 or the composition for administration of any one of items A44-A56 or the feed, feed additive or premix according to any of items A57-59 for:
preventing growth or decreasing the number, of E. coli in the gastrointestinal tract and/or
for increasing, maintaining or re-establishing dry matter content of faeces for piglets with post-weaning diarrhoea compared to post-weaning piglets with PWD that did not consume the composition.

Item B7. The composition according to any one of claims items A1-A43 or the composition for administration of any one of items A44-A56 or the feed, feed additive or premix according to any of items A57-59 for use in preventing and/or treating post-weaning diarrhoea in a piglet.

Item B8. Use of the composition according to any of items A1-A43 or the composition for administration of any one of items A44-A56 or the feed, feed additive or premix according to any of items A57-59 for feeding weaning piglets for 0-5 weeks post-weaning.

Item B9. Use of the composition according to any of items A1-A43 or the composition for administration of any one of items A44-A56 or the feed, feed additive or premix according to any of items A57-59 for improving or maintaining the biophysical characteristics of animals compared to animals that do not receive the composition.

Item B10. The use according to item B9 wherein the biophysical characteristic is at least one characteristic selected from the group consisting of:
a) An increase in feed conversion ratio
b) An increase in weight
c) An increase in drinking water consumption
d) An increase in the dry matter content of faeces,
e) A decrease in symptoms of PWD
f) An increase in feed consumption.

Item B11. The use according to item B10, wherein the increase in weight is in the range of 1%-100% per week.

Item B12. The use according to any one of items B10 or B11 wherein the increase in weight is at least 0.5-1.3 kg per animal per week.

Item B13. Use of the composition of any one of items A1-A43 or the composition for administration of any one of items A44-A56 or the feed, feed additive or premix according to any of items A57-59 for increasing weight gain in weaning piglets relative to weaning piglets who do not consume the composition.

Item B14. Use of the composition of any one of items A1-A43 or the composition for administration of any one of items A44-A56 or the feed, feed additive or premix according to any of items A57-59 for increasing the dry matter content in faeces of piglets with post-weaning diarrhoea compared to the post-weaning piglets with PWD that do not consume the composition.

Item B15. Use of the composition of items A1-A43 or the composition for administration of any one of items A44-A56 or the feed, feed additive or premix according to any of items A57-59 for maintaining or re-establishing dry matter content of faeces for piglets with post-weaning diarrhoea.

Item B16. Use of the composition of any one of items A1-A43 or the composition for administration of any one of items A44-A56 or the feed, feed additive or premix according to any of items A57-59 for preventing growth of E coli in the gastrointestinal tract.

Item B17. Use of the composition of any one of items A1-A43 or the composition for administration of any one of items A44-A56 or the feed, feed additive or premix according to any of items A57-59 to decrease the number of pathogenic bacteria in the gastrointestinal tract.

Item B18. The composition of any one of items A1-A43 or the composition for administration of any one of items A44-A56 or the feed, feed additive or premix according to any of items A57-59 for use as a medicament.

Item B19. The composition of any one of items A1-A43 or the composition for administration of any one of items A44-A56 or the feed, feed additive or premix according to any of items A57-59 for use in preventing and/or treating post-weaning diarrhoea in a piglet.

Item B20. The composition of any one of items A1-A43 or the composition for administration of any one of items A44-A56 or the feed, feed additive or premix according to any of items A57-59 for use in improving resistance to entero toxigenetic E coli bacteria in weaning piglets compared to weaning piglets that do not consume the composition.

Item B21. A method of preventing and/or treating post-weaning diarrhoea in a piglet by administering the composition of any one of items A1-A43 or the composition for administration of any one of items A44-A56 or the feed, feed additive or premix according to any of items A57-59.

Item B22. Use of the composition of any one of items A1-A43 or the composition for administration of any one of items A44-A56 or the feed, feed additive or premix according to any of items A57-59 in combination with a feed free from medical zinc.

Item B23. Use of the composition of any one of items A1-A43 or the composition for administration of any one of items A44-A56, the feed, feed additive and/or premix of any of items A57-A59 for reducing the use of antibiotics and/or medical zinc oxide while increasing or maintaining the biophysical characteristic of weaning piglets.

Item B24. The composition of any of items A1-A43 or the composition for administration of any one of items A44-A56 or the feed, feed additive or premix according to any of items A57-59 for the use of any of items B1-B23, wherein an effective amount of binding agent is consumed.

Item B25. The use of item B24, wherein an effective amount of binding agent is least 0.01 g binding agent per kg body weight per day, for example 0.1 g, 0.2 g or 0.3 g.

Item B26. The use of item B24-B25 wherein the composition for administration is administered via drinking water, and wherein 1.5-5 L of the drinking water is consumed per animal per day.

Item C1. A method of preparing the composition of any one of items A1-A43 or the composition for administration of any one of items A44-A56, said method comprising the steps of:
a) Combining acid component(s), a binding agent; and
b) obtaining a composition.

Item C2. The method of item C1, said method comprising the steps of:
a) Combining acid component(s), a binding agent, and a liquid; and
b) obtaining a composition.

Item C3. The method of item C1 or C2 said method comprising the steps of:
a) Combining acid component(s), a binding agent, and a liquid; and
b) obtaining a composition for storage/transport.

Item C4. The method of item C1-C3 said method comprising the steps of:
a) Combining acid component(s), a binding agent, and a liquid; and
b) obtaining a composition for administration.

Item C5. A method of preparing a composition for administration comprising the steps of:
a) combining an acid component, a binding agent and a liquid
b) obtaining a composition for storage/transport
c) diluting the composition for storage/transport
d) obtaining a composition for administration.

Item C6. A method of preparing the composition of any one of items A1-A43 or the composition for administration of any one of items A44-A56 or the feed, feed additive or premix according to any of items A57-59, said method comprising the steps of:
a) providing a feed comprising a protein source, a carbohydrate source, a lipid source and optionally one or more ingredients selected from the group consisting of fish meal, vitamins, mineral premix and combinations thereof,
b) providing acid component(s) and a binding agent, wherein the binding agent comprises a tannin component and/or a polygalactose component, and
c) obtaining a composition comprising acid component(s); and a tannin component and/or a polygalactose component.

Item D1. A method of administering the composition of any one of items A1-A43 by adding the composition to drinking water to form a composition for administration according to items A44-A56.

Item D2. A method of administering the composition any of one of items A1-A43 to an animal in need thereof, said method comprising the steps of;
a) Combining the composition of any one of items A1-A43 with drinking water,
b) Obtaining drinking water comprising the composition for administration according to any one of items A44-A56, the composition for administration,
c) providing the animal in need thereof with the drinking water obtained in step b).

Item D3. A method of administering the composition of any one of any one of items A1-A43 to an animal in need thereof, said method comprising the steps of;
a) Combining the composition of the invention with drinking water,
b) Obtaining drinking water comprising a composition for administration according to any one of items A44-A56, comprising:
   an amount of lactic acid component between 0.0054%-5.4% w/w,
   an amount of acetic acid component between 0.00131%-0.131% w/w,
   an amount of tannin component, when present, between 0.00259%0.259% w/w,
   and an amount of polygalactose component, when present, between 0.000259%-0.0259% w/w, and
c) providing the animal in need thereof with the drinking water obtained in step b).

Item D4. The method of any one of item C1-C5 wherein the drinking water comprising the composition for administration according to any one of items A44-A56 is administered via a dosing chamber.

Item D5. A method of administering a liquid composition according to item A43, or a liquid composition for administration according to items A44-A56, the method comprising;
pouring the liquid composition over a dry feed, wherein the liquid composition is poured over the dry feed at least twice every 24 hours, wherein at least 0.2 L of the liquid composition is provided per piglet twice a day, and wherein the dry feed is free from medical zinc.

Item D5. A method of administering the composition wherein the composition for administration of any of items A44-A56 is in the form of drinking water, and wherein an animal in need thereof consumes and effective amount of binding agent by consuming the drinking water.

Item D6. The method of item D5 wherein an effective amount of binding agent is at least 0.01 g binding agent per kg body weight per day, for example 0.1 g, 0.2 g or 0.3 g.

Item D7. The method oo any of items D5 or D6 wherein the animal in need thereof consumes an effective amount of binding agent per kg body weight per day by consuming between 1.5 L and 5 L of the composition for administration per day, wherein the composition for administration is administered via drinking water.

Item D8. A method of feeding and/or watering an animal comprising administering to said animal the composition for administration according to any one of items A44-A56.

### EXAMPLES

### Example 1: Formulation

### Example 1.1: A Composition Comprising Lactic Acid and Acetic Acid Added Directly to Dry Feed

The objective if this experiment was to investigate the effect of adding the feed additive in the first week after weaning. A batch of water solution was prepared using liquid Lactic Acid (80% Lactic Acid, Frederiksen, DK) and Acetic Acid (5%, Organic Vinegar, Netto, DK) corresponding to a content of 1.0% Lactic Acid and 0.02% Acetic Acid.

The feed used at the pig farm consisted of 50% Wheat, 15% Barley, 10% Dry Fish Flour, 3% Soya Oil, 10% “Heavy Pig 3” conc. 20%. The components of the feed were bought at local suppliers in North Zealand, Denmark. The feed was free from medical zinc.

### Purpose

To analyse whether the added components can increase the overall growth of the piglets in the test group including the ability to reducing the occurrence of diarrhoea.

### Set Up

Examples 1 consisted of a one week feeding experiment with day-one weaned piglets. A water solution of 1.0% Lactic Acid and 0.02% Acetic Acid (described above) was poured onto the dry feed of the test group twice a day, corresponding to 0.25 L per pig twice per day. The weight gain in both groups was measured on the first day of receiving the composition (Day 0), and one week later (Day 7).

### Results

TABLE 1

| | | Test | Control | |
|---|---|---|---|---|
| Date | | group | group | Comments |
| 6 Sep. | Weight total | 91 | 94 | Day 0 |
| 2019 | (kg) | | | |
| | Weight avg (kg) | 5.4 | 5.5 | |
| | No. of piglets | 17 | 17 | |
| 13 Sep. | Weight total | 113 | 106 | Day 7 |
| 2019 | (kg) | | | |
| | Weight avg (kg) | 6.6 | 6.2 | |
| | No. of piglets | 17 | 17 | |
| | Weight gain | 22 | 12 | (Total end |
| | total (kg) | | | weight minus |
| | | | | total start |
| | | | | weight) |
| | Average | 1.3 | 0.7 | (Weight gain |
| | weight gain | | | total)/(number |
| | (AWG) | | | of piglets) |
| | (kg/piglet) | | | |

Ratio AWG: Test group/Control group = 1.85

### Conclusion of Example 1.1

The applied composition of the feed additive provides a high weight gain in the test group compared to the control group (1.3 vs. 0.7 kg/piglet, or an 85% higher weight gain for piglets that received the test composition of Example 1.1).

Diarrhoea was not observed in either group. However, the risk for getting diarrhoea is reduced gradually as a piglet grows. This means that increasing growth rate for piglets in the test group reduces their risk of diarrhoea compared to piglets in the control group.

### Example 1.2: Administration of Lactic Acid and Acetic Acid Via Drinking Water

In order to increase robustness, we decided to investigate whether the composition would have an effect if administered through drinking water instead of adding it by spreading on the feed twice daily. Two independent one-week feeding experiments with day-one weaned piglets, were conducted (Examples 1.2.1 and 1.2.2 respectively). In the experiments, a composition comprising lactic acid and acetic acid, and no tannin was administered to the piglets.

A batch solution was prepared using liquid lactic acid (80%) lactic acid, Frederiksen, DK) and acetic acid (5%, Organic Vinegar, Netto, DK) corresponding to a content of 1.0% lactic acid and 0.02% acetic acid.

TABLE 2

| | | Concentration (kg/L) | Concentration in |
|---|---|---|---|
| | | in stock for Dosatron | drinking water |
| Compound | Supplier | dosing at 2% | (% w/v) |
| Lactic acid | Frederiksen A/S | 0.17 | 0.14 |
| (80% v/v) | | | |
| Acetic acid | Organic vinegar, | 0.0036 | 0.003 |
| (5% v/v) | Netto A/S | | |

### Purpose

In has been described that the switch from a liquid to a solid diet damages intestinal villi, with an impairment in intestinal absorption and secretion of digestive enzymes, thereby leading to a reduced growth rate (Bonetti et al 2021).

To analyze whether the effect of the added components is comparable or improved when administered through the drinking water and can increase the overall growth of piglets including the ability to reduce the occurrence of diarrhoea.

### Set Up

Two independent one-week feeding experiments with day-one weaned piglets were carried out. A concentrated 10 times solution containing 1.7% lactic acid and 0.036% acetic acid (described above) was prepared and used to mix with the standard setup for drinking water used at the farm by the weaned pigs.

Administration of the feed additive comprises 0.14% lactic acid and 0.0035% acetic acid in the drinking water for one to two weeks was performed in two independent experiments. In each experiment, two test groups were administered the composition while two control groups received tap water. All test and control groups had free access to their respective drinking water.

The total weight of all piglets in each group was measured at the initiation of the experiment (one day after weaning (Day 0), “Start total weight”) and after one week (Day 7) and two weeks (Day 14) as indicated (“End total weight”).

It is generally known that the amount of drinking water taken up by weaned pigs correlates with the amount of feed consumed by the piglet. To ensure constant administration of lactic acid and acetic acid in the drinking water for the test groups, a water-powered proportional dosing pump that is used to e.g., administer medication in farms (Dosatron) was calibrated to administer drinking water comprising 0.14% lactic acid and 0.002% acetic acid. The feed was as described in the above experiments, and contained the standard amount of zinc (1.5-2.5 g/kg feed) normally used to reduce the risk and spread of infection. Following the one-week administration, the total weight gain of each group was monitored. For each experiment, two independent groups (Test group 1 and 2) and two control groups (Control 1 and 2) were included. Unless indicated, each group included 16 piglets.

### Results

In Example 1.2.1, a clear increase in the average weight gain (AWG) was observed. No diarrhoea was observed for any of the control or test groups in the experiments.

TABLE 3
Average weight gain for piglets (Day 14) in Experiment 1.2.1.

| | | | Control | Control |
|---|---|---|---|---|
| | Test 1* | Test 2 | 1* | 2 |
| Start total weight (kg) (Day 0) | 100.5 | 90.5 | 106.0 | 93.0 |
| End total weight (kg) (Day 14) | 116.5 | 107.5 | 119 | 104 |
| Total weight gain (kg) | 16.0 | 17.0 | 13.0 | 11.0 |
| Average weight gain | 1.07 | 1.06 | 0.87 | 0.69 |
| (AWG) (kg/piglet) | | | | |

*Test and control groups included 16 piglets except for in Experiment 1.2.1 where Test 1 and Control 1 included 15 piglets.
Ratio average weight gain Experiment 1.2.1: (Experiment 1.2.1 + Experiment 1.2.2)/(Control 1.2.1 + Control 1.2.2) = 1.37
Ratio average weight gain Experiment 1.2.2 = (1.36).

### Conclusion of Example 1.2

Administration of lactic acid and acetic acid via drinking water provided improved weight gain for piglets (Table 3). No diarrhoea was observed. These results indicate that administration of a composition comprising lactic acid and acetic acid via drinking water provides consistent weight gain when administered.

### Example 1.3. Administration of Lactic Acid, Acetic Acid and a Tannin Component Via Drinking Water

A concentrated stock solution is required to facilitate administration through drinking water. Several attempts using albumin-tannate in a concentrated solution revealed problems with solubility, resulting in non-homogeneous solutions comprising a precipitate. The tannin in Experiment 1.3 is tannic acid, which has a high solubility in acidic solutions.

In Example 1.3, a composition comprising liquid lactic acid, liquid acetic acid and tannic acid was administered to piglets in combination with a feed free from medical zinc.

A batch solution was prepared using liquid lactic acid (80% lactic acid, Frederiksen, DK); acetic acid (5%, Organic Vinegar, Netto, DK) and tannic acid (Sigma) corresponding to a content of 10% Tannic acid, 69% lactic acid and 1.4% acetic acid. This composition is hereafter named TLA stock (tannic acid, lactic acid and acetic acid stock).

TABLE 4

| | | Concentration (kg/L) in | Concentration in |
|---|---|---|---|
| | | stock for Dosatron | Drinking water |
| Compound | Supplier | dosing at 2 % | (% w/v) |
| Lactic acid | Frederiksen A/S | 0.17 | 0.14 |
| (80% v/v) | | | |
| Acetic acid | Organic vinegar, | 0.0036 | 0.003 |
| (5% v/v) | Netto A/S | | |
| Tannic acid | Sigma | 0.025 | 0.02 |
| (100% w/v) | | | |

### Purpose

To analyse whether administering a composition comprising of lactic acid, acetic acid and tannic acid improves overall weight gain of piglets when administered via drinking water. The purpose is also to analyse whether tannic acid can be used as a replacement for medical zinc in piglet feed, for example by reducing the occurrence of diarrhoea.

### Set Up

Two independent one-week feeding experiments were carried out with day-one weaned piglets (Example 1.3.1 and 1.3.2).

The concentrated TLA stock feed additive was prepared and used to mix with the standard setup for drinking water used by the weaned pigs at the farm. The dry feed is as described above.

The feed received by both the test and control groups was free from medical zinc. In each experiment, two test groups were administered the TLA composition in drinking water while two control groups received standard tap water. The total weight of all piglets in each group was measured at the initiation of the experiment (Day 0, “Start total weight”) and after one (Day 7) and two weeks (Day 14) as indicated (“End total weight”). Light levels of diarrhoea were observed in all groups. In each experiment, two test groups were administered the composition containing lactic acid, acetic acid and tannic acid whilst two control groups received tap water.

Following one-week administration, the total weight gain of each group was monitored. For each experiment, two independent test groups (Test 1 and Test 2) and two control groups (Control 1 and Control 2) were included. All groups included 16 piglets.

### Results

Light levels of diarrhoea were observed in all groups, consistent with the lack of zinc in the supplied feed to all groups.

In Example 1.3.2, two piglets of Control group 2 developed clear signs of diarrhoea on day 4 and were treated with antibiotics for ethical reasons. Accordingly, this group showed the lowest average weight gain per piglet (0.38 kg).

Despite variation in the start weight between Example 1.3.1 and 1.3.2, a similar weight gain was obtained (with a 64-77% increase), indicating a consistent effect in growth gain in a feed substantially free of zinc and comprising tannic acid.

The higher AWG ratio observed compared to Example 1.2. The results indicate an additional effect of tannic acid in the increase in weight in an environment where signs of light diarrhoea were observed. The increase in weight gain is comparable to the highest ratio observed in piglets that were (i) fed with a composition comprising lactic acid and acetic acid (no tannic acid) in combination with a feed comprising zinc, and (ii) where the inventive composition (without tannic acid) was poured over the dry feed and not administered through the drinking water (Examples 1.1 and 1.2).

TABLE 5
Example 1.3.1

| | Test | Test | Control | Control |
|---|---|---|---|---|
| | group 1 | group 2 | group 1 | group 2 |
| Start total | 105.0 | 83 | 99.0 | 83.0 |
| weight (kg) | | | | |
| End total weight | 118.0 | 93.0 | 106.5 | 89.5 |
| (kg) | | | | |
| Weight gain | 13 | 10.0 | 7.5 | 6.5 |
| (kg) | | | | |
| Average | 0.81 | 0.63 | 0.47 | 0.31 |
| weight gain | | | | |
| (AWG) | | | | |
| (kg /piglet) | | | | |

Ratio AWG Example 1.3.1: (Test 1 + Test 2)/(Control 1 + Control 2) = 1.64

TABLE 6
Example 1.3.2

| | Test | Test | Control | Control |
|---|---|---|---|---|
| | group 1 | group 2 | group 1 | group 2 |
| Start total | 95.5 | 86.0 | 101.5 | 89.0 |
| weight (kg) | | | | |
| End total weight | 108.5 | 96.0 | 108.5 | 95.5 |
| (kg) | | | | |
| Weight gain | 13 | 10.0 | 7.0 | 6.0 |
| (kg) | | | | |
| Average | 0.81 | 0.63 | 0.44 | 0.38 |
| weight gain | | | | |
| (AWG) | | | | |
| (kg/piglet) | | | | |

Ratio AWG Example 1.3.2: (Test 1 + Test 2)/(Control 1 + Control 2) = 1.77

Tables 5 and 6 show that administration of the feed additive containing lactic acid, acetic acid and tannic acid in the drinking water for one week to weaned piglets in the absence of zinc results in weight gains in independent experiments. Each test and control group consisted of 16 piglets. Two of the piglets in the Control group 2, Example 1.3.2 showed clear signs of diarrhoea at day 4 and needed intensive antibiotic treatment.

### Conclusion of Example 1.3

The TLA composition comprising tannic acid, lactic acid and acetic acid provided consistent weight gains in two independent experiments (Tables 5 and 6) demonstrating that the effect of the administration of the composition is obtained when provided in the drinking water. The use of tannic acid results in high weight gains in piglets fed without the use of zinc.

The use of the TLA feed additive results not only in the prevention of disease but also in a consistently high level of weight gain. The TLA feed additive composition may effectively substitute the use of zinc in the feed.

Further improvements in the ratios of weight gains can be envisaged by further adjusting the concentration of the components of the TLA feed additive in dose/response trials and extending its administration to weaned piglets for 4-7 weeks.

### Conclusion of Example 1

The results of Examples 1.1-1.3 show that a composition comprising lactic acid, acetic acid and tannic acid is effective in improving or maintaining weight gain in post-weaning piglets.

The results further show that the composition is effective when administered via drinking water. Administration via drinking water allows for controlled dosing and is easy for farmers to implement.

### Example 2: Protection Against Infection

The two main pathogens associated with post-weaning diarrhoea (PWD) in piglets are enterotoxigenic Escherichia coli (ETEC) expressing F4 or F18 fimbriae (Luise et al, 2019). Antimicrobial resistance (AMR) has globally been acknowledged as a serious threat to human health and boosted research into developing nutritional and feeding strategies as well as vaccination protocols to combat PWD due to ETEC. Zinc, often in the form of ZnO, is often added to piglet feed in order to prevent/treat PWD. In view of the use of the use of zinc being outphased in the EU, alternatives are needed.

An established approach to investigate the potency of pro-biotic and pre-biotic substances for preventing PWD is to apply a piglet in vivo challenge model with ETEC infection.

### Purpose

To investigate the effect of the inventive composition on piglets with PWD, a standardized challenge experiment was carried out. The experiment was performed at Department of Animal Science, University of Aarhus, the only Danish Institution recognized and accredited capable of performing challenge experiments in piglets. The experiment involved weaning pigs that are sensitive to ETEC F18.

### Set Up

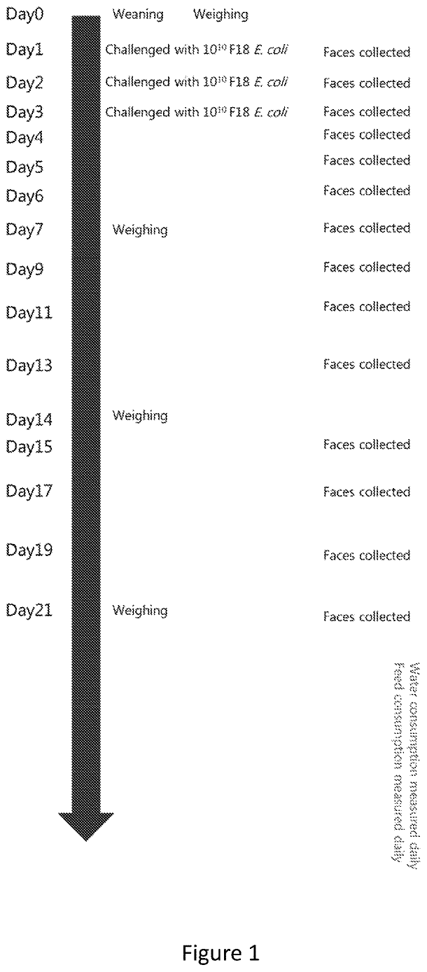

An overview and timeline for the F18 challenge experiment is shown in FIG. 1.

Two groups, each of 10 pigs, were included in the experiment. The test group received the composition as described in Example 1.3 (final concentrations 0.64% (w/v) lactic acid; 0.0131% (w/v) acetic acid; 0.0925% (w/v) tannic acid), administered via drinking water as described in Example 1.3, with dosing set to 3.7%.

The test group had access to the drinking water including lactic acid, acetic acid and tannic acid, and the control group only had access to tap water. All groups had unrestricted access to the respective water.

For both groups, two pigs were kept in each pen. Pigs were weaned at day 0 and the test group was challenged with three consecutive doses of 10¹⁰ F18 E. coli on day 1, 2, and 3 after weaning. Feed consumption and water consumption were registered once a day with 24 hours intervals. Faeces samples were collected once a day for dry matter quantification, while the weight of each pig was determined every seven days starting from Day 0.

All test and control group were fed with the same dry feed as shown in Table 7.

TABLE 7

| Component | % |
|---|---|
| Wheat | 52.26 |
| Barley | 15.08 |
| Soybean meal | 12.00 |
| Soybean concentrate | 4.75 |
| Whey powder | 4.00 |
| Potato protein | 3.00 |
| Soybean oil | 2.50 |
| Fish meal 70% | 2.00 |
| Dicalcium phosphate | 1.83 |
| Salt (NaCI) | 0.40 |
| Limestone (CaCO3) | 0.78 |
| Lysine-HCI | 0.54 |
| Methionine DL | 0.22 |
| Threonine | 0.20 |
| Tryptophan | 0.07 |
| Valine L | 0.04 |
| Vitamin Premix | 0.20 |
| Phytase | 0.03 |
| Luctarom | 0.10 |

### Dosing:

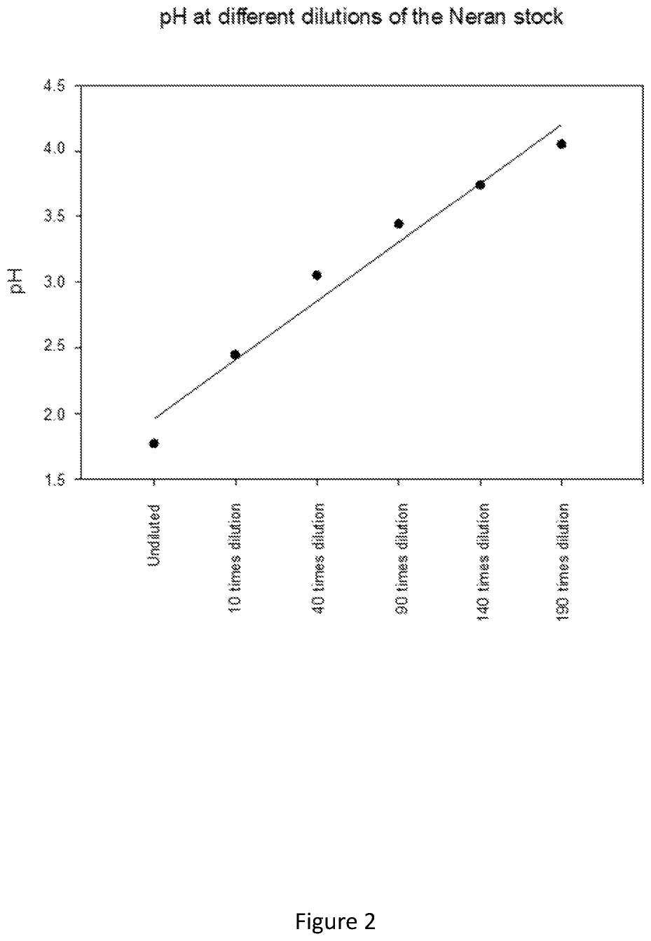

It is important to find a way to dose the test composition in a consistent manner, and as such, a Dosatron® (France) was used. Daily quality control tests were carried out to ensure that the drinking water administered to the test groups was dosed correctly. FIG. 2 shows that measuring pH in the water that the pigs receive confirms that dosing of the product is correct.

The data in Table 8 show the pH of the drinking water administered to the test group over 14 days. Drinking water samples were collected daily from each pen (each hosting two pigs). The pH was measured with a pH strip and a transportable pH electrode. The data show that variations in the measured pH value are very low, reflecting that each pen consistently received the intended dose over the two-week period.

TABLE 8
pH of drinking water administered to test subjections.

| Days post | Pen | | | | |
|---|---|---|---|---|---|
| weaning | 67 | 69 | 71 | 74 | 76 |
| 0 | 2.7 | 2.8 | 2.9 | 3 | 3.2 |
| 1 | 3.1 | 2.8 | 2.9 | 2.8 | 2.8 |
| 2 | 3.1 | 3.4 | 3 | 2.9 | 2.9 |
| 3 | 3.2 | 3.4 | 3.1 | 3.2 | 3.2 |
| 4 | 2.9 | 2.9 | 2.8 | 2.9 | 2.9 |
| 5 | 2.9 | 2.9 | 2.8 | 2.9 | 2.8 |
| 6 | 2.7 | 2.8 | 2.8 | 2.7 | 2.7 |
| 7 | 3 | 2.9 | 2.9 | 2.9 | 2.9 |
| 8 | 2.9 | 3 | 3 | 2.8 | 2.9 |
| 9 | 3 | 2.9 | 2.9 | 2.9 | 2.9 |
| 10 | 2.9 | 2.8 | 2.9 | 2.8 | 2.8 |
| 11 | 2.9 | 2.9 | 2.9 | 2.9 | 2.8 |
| 12 | 2.9 | 2.9 | 2.8 | 2.8 | 2.9 |
| 13 | 2.8 | 3 | 2.8 | 2.8 | 2.8 |
| 14 | 3 | 2.9 | 2.8 | 2.9 | 2.9 |
| Mean pH | 2.9 | 3.0 | 2.9 | 2.9 | 2.9 |

### Results

### Success of the Challenge Experiment:

A precise indicator for a successful challenge experiment is increased shedding of the infectious F18 ETEC strain from the pig intestine. The content of F18 E. coli in faeces samples from test piglets and control piglets are shown in FIG. 3.

It is evident from the data that both groups of pigs showed a peak of ETEC F18s in the faeces after administration in accordance with what has been reported before (Nadeau et al 2017). It can therefore be concluded that the infection challenge was successful. It is also evident from the data in FIG. 3 that the pigs offered the test composition in the drinking water only shows a transient peak in the F18 ETEC content at day 6 while the control group already shows a peak after three days and that the F18 content remains high for a longer period of time indicating that the F18 ETEC cells reside for a longer period of time in the intestine of the pigs only having access to common drinking water. The test composition therefore has a positive effect on preventing F18 ETEC cells from colonizing the intestine.

### Dry Matter Content:

Dry matter content in faeces is a universally accepted indicator of the extent of diarrhoea. For determination of dry matter content, faeces were collected from the pens, and weighed. The collected samples were freeze dried and the water content was evaporated via heat treatment. The mass of the resulting solid material was then determined, and the dry matter content calculated. Dry matter percentage=100*mass after water evaporation/mass before water evaporation.

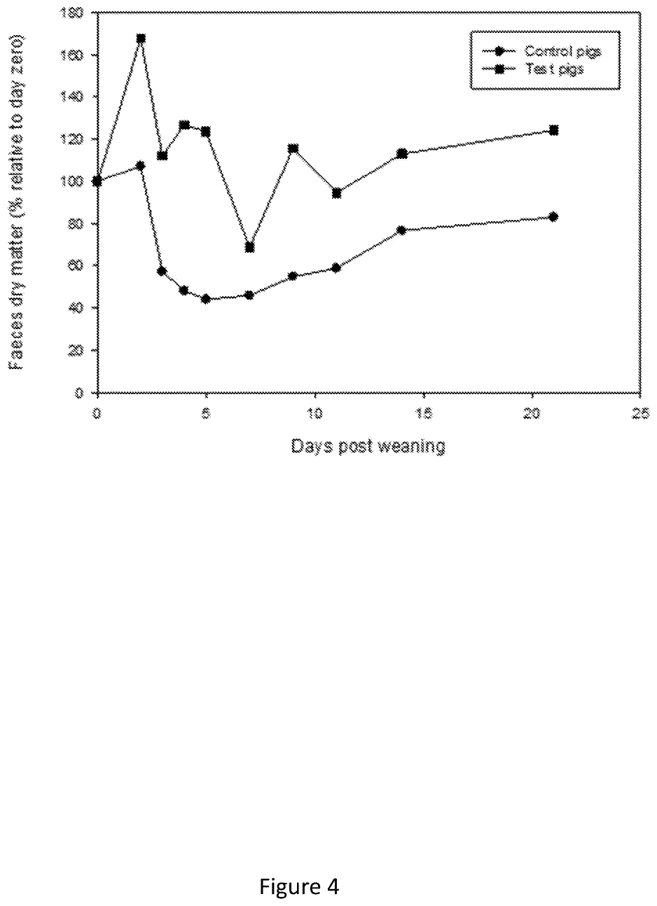

FIG. 4 shows the development in dry matter content in faeces from the test group and for the control group.

Diarrhoea occurred in the control piglets at day 2, and for three of the pigs lasted for up to day 10.

In contrast, most of the test piglets showed a low dry matter content at day 7, which is indicative of diarrhoea, but recovered very fast to a dry matter content resembling that of day 0.

In conclusion, the challenge experiment shows that the composition of the invention postponed the onset of diarrhoea observed in the test group.

The test group also overcame diarrhoea more quickly than the control group, as seen by the dry matter content increasing more rapidly after infection. The dry matter content of the test group also returned to that observed before the F18 challenge was initiated (Day 0), whereas the dry matter content of the control group did not return to that of Day 0.

### Feaces Score:

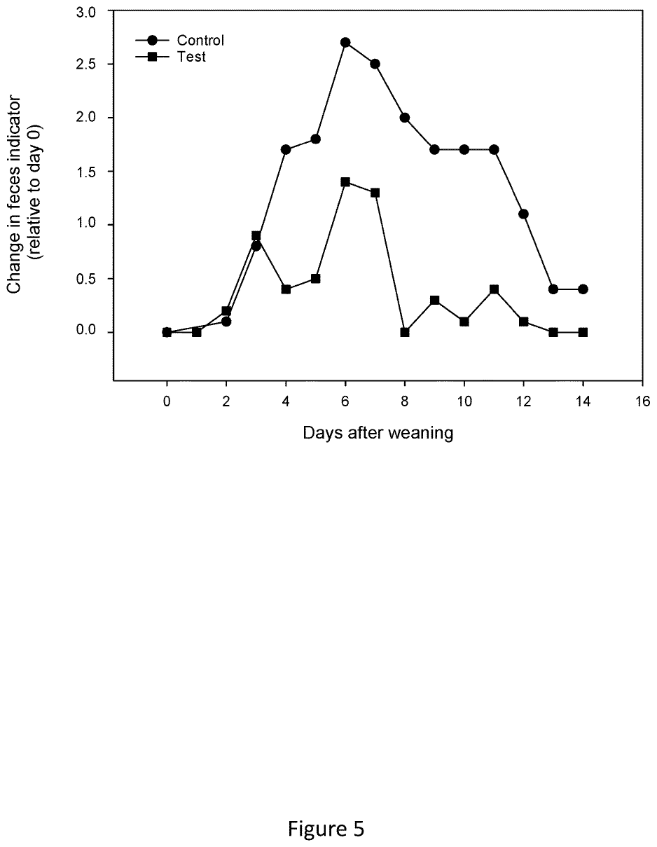

An alternative way to score the diarrhoea is to evaluate the faeces directly in the pen. Data from the test group of pigs and the control group are depicted in FIG. 5 and show that the test group gets a lower score than the control group-indicative of less diarrhoea.

### Conclusion of Example 2.1

Example 2.1 shows that the administration of lactic acid, acetic acid and tannic acid to piglets provides protection against ETEC F18 infection. It can be concluded that the lactic acid, acetic acid and tannic acid in drinking water can therefore effectively substitute the use of antibiotics and zinc in pig farming.

### Example 2.2: Diarrhea

The two main pathogens associated with post-weaning diarrhoea (PWD) in piglets are enterotoxigenic Escherichia coli (ETEC) expressing F4 or F18 fimbriae (Luise et al, 2019). Antimicrobial resistance (AMR) has globally been acknowledged as a serious threat to human health and boosted research into developing nutritional and feeding strategies as well as vaccination protocols to combat PWD due to ETEC.

Zinc, often in the form of ZnO, is often added to piglet feed in order to prevent/treat PWD. In view of the use of the use of zinc being outphased in the EU, alternatives are needed.

An established approach to investigate the potency of pro-biotic and pre-biotic substances for preventing PWD is to apply a piglet in vivo challenge model with ETEC infection.

### Purpose

To investigate the effect of the inventive composition on piglets with PWD, a standardized challenge experiment was carried out. The experiment was performed at Department of Animal Science, University of Aarhus, the only Danish Institution recognized and accredited capable of performing challenge experiments in piglets. The experiment involved weaning pigs that are sensitive to ETEC F18.

### Set Up

An overview and timeline for the F18 challenge experiment is shown in FIG. 1.

Two groups, each of 10 pigs, were included in the experiment. The test group received a composition with final concentrations of 0.6682% (w/w) lactic acid; 0.014% (w/w) acetic acid; 0.0259% (w/w) tannic acid: 0.00259% (w/w) polygalactose component), administered via drinking water as described in Example 1.3, with dosing set to 3.7%.

The test group had access to the drinking water including lactic acid, acetic acid, tannic acid and polygalactose, and the control group only had access to tap water. All groups had unrestricted access to the respective water.

For both groups, two pigs were kept in each pen. Pigs were weaned at day 0 and the test group was challenged with three consecutive doses of 0.5·10¹⁰) F18 E. coli on day 1, 2, and 3 after weaning for experiments wherein the composition comprise lactic acid, acetic acid and tannic acid, and on day 1 and day 2 for experiments wherein the composition comprised lactic acid, acetic acid, tannic acid and polygalactose. Feed consumption and water consumption were registered once a day with 24 hours intervals. Faeces samples were collected once a day to determine the number of pigs with diarrhea.

All test and control group were fed with the same dry feed as used in Table 7.

### Dosing:

It is important to find a way to dose the test composition in a consistent manner, and as such, a Dosatron® (France) was used. Daily quality control tests were carried out to ensure that the drinking water administered to the test groups was dosed correctly.

### Results

### Success of the Challenge Experiment:

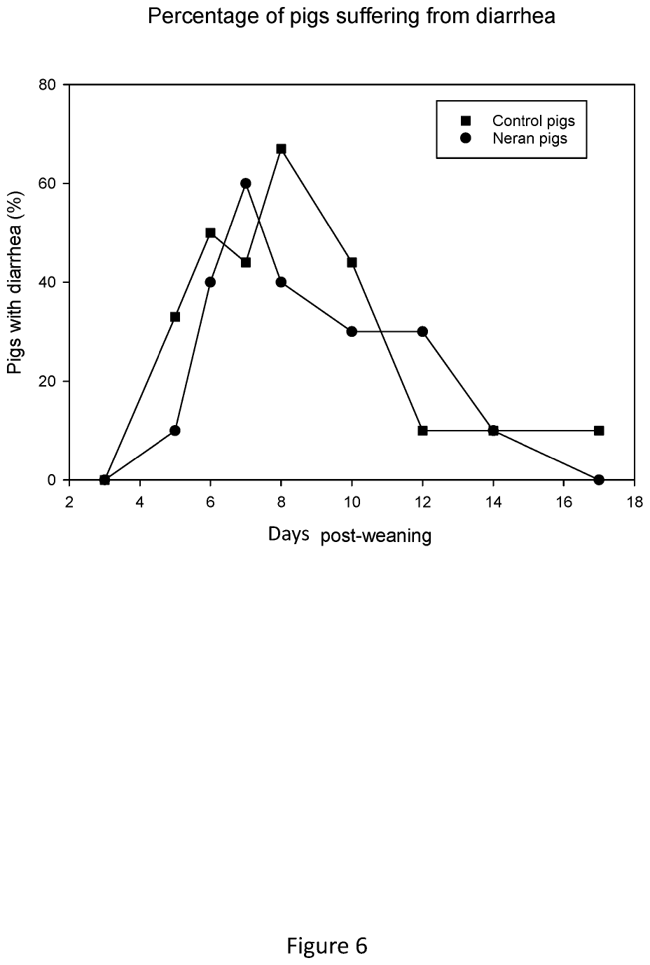

An indicator for a successful experiment is a decrease in infected pigs when compared to the control group. The percentage of pigs suffering from diarrhea is shown in FIG. 6.

It is evident from the data that both groups of pigs showed an increase in percentage of pigs with diarrhea the first few days after infection. It can therefore be concluded that the infection was successful.

It is also evident from the data in FIG. 6 that the pigs offered the test composition in the drinking water are less likely to get diarrhea from the infection and are also more likely to get cured after 17 days.

The test composition therefore has a positive effect on preventing pigs from suffering from diarrhea.

### Conclusion of Example 22

Example 2.2 shows that the administration of lactic acid, acetic acid, tannic acid and polygalactose to piglets provides protection against diarrhea. It can be concluded that the lactic acid, acetic acid, tannic acid and polygalactose in drinking water can therefore effectively substitute the use of antibiotics and zinc in pig farming.

### Example 3.1: Water and Feed Intake

It is well known a weaning piglet's food and water consumption is positively correlated, and as such, it is beneficial for weaning piglets to eat and drink at high volumes.

The benefits of drinking at high volumes are further enhanced in this instance due to the drinking water composition comprising lactic acid, acetic acid and tannic acid which is shown to provide protection against infection in Example 2.1.

Lactic acid is known to have an unpleasant taste. The drinking water comprising lactic acid, acetic acid and tannic acid received by the test piglets should therefore be palatable so that the piglets consume a comparable amount of water to the control piglets.

### Purpose

The purpose of Example 3.1 is to analyse whether the drinking water comprising lactic acid, acetic acid and tannic acid is palatable to the piglets. The piglets should consume a volume of feed and water such that the beneficial effects of the drinking water additives are realized.

### Set Up

The content of the feed received by the test group piglets is shown in Table 7. The content of the drinking water received by the test group piglets is shown in Table 9. Drinking water for the test group piglets was administered via Dosatron (Dosatron©, France) at a setting of 3.7%. Test group piglets were administered only tap water.

TABLE 9
Composition of the drinking water administered by test group piglets.

| | Lactic acid | Acetic | Tannic |
|---|---|---|---|
| | (80%) | acid (5%) | acid (100%) |
| Stock solution | 3.6 L (0.69 kg | 1.4 L (0.014 kg | 500 g (0.1 kg |
| | LA/L) | HAc/L) | TA/L) |
| 1 + 3 dilution | 0.17 kg LA/L | 0.0036 kg HAc/L | 0.025 kg/L |
| Fed to test pigs | 0.0064 kg LA/L | 0.00013 kg/L | 0.0009 kg/L |

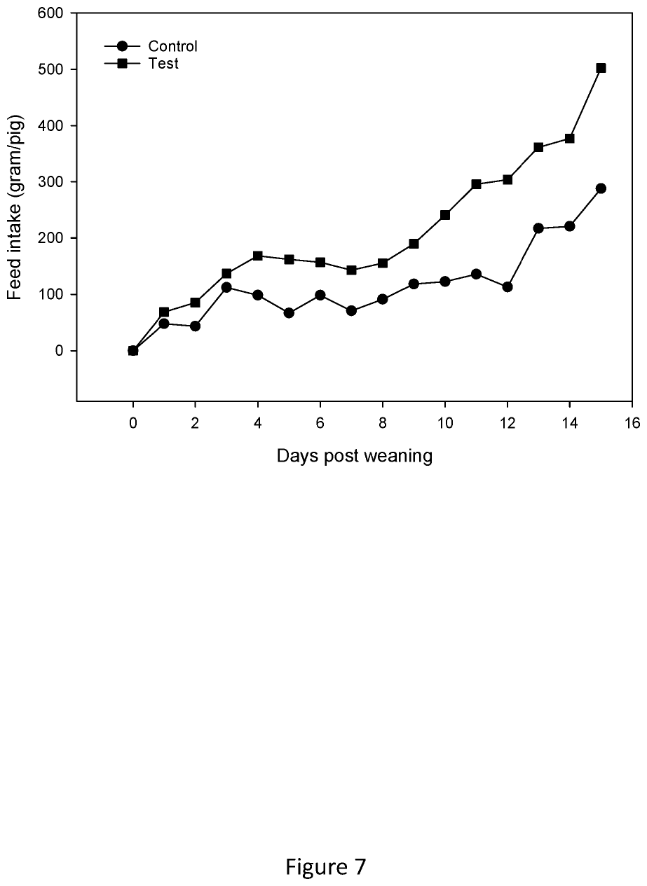

The feed intake of the control and test piglets was measured every day from day 0 to day 16, and the average feed intake calculated. Results are show in in FIG. 7.

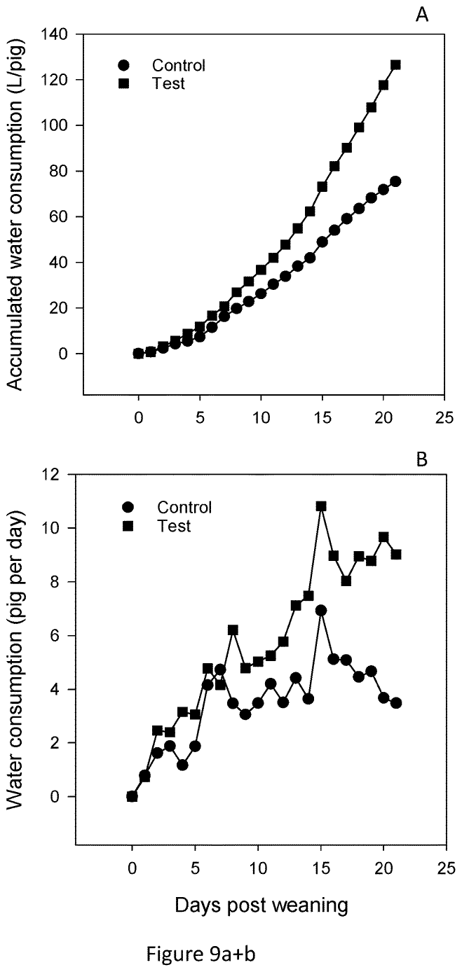

The average water intake of the test and control piglets was measured every day from Day 0 to Day 21, and the accumulated water intake calculated. Results are shown in FIGS. 9a and 9b.

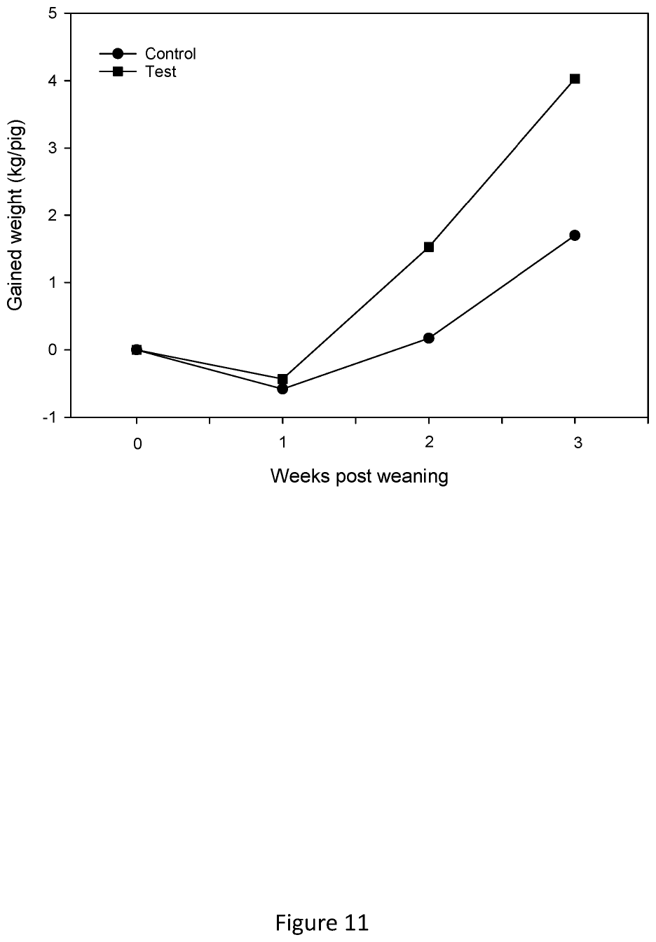

The test and control piglets were individually weighted on Day 0, Day 7, Day 15 and Day 21, and the average weight gain calculated. Results are shown in FIG. 11.

### Results

### Feed Intake:

The feed consumed per pig during the challenge experiment of Example 2 is shown in FIG. 7. FIG. 7 shows that the test group piglets consumed considerable more feed than the control group piglets. In accordance with the experiments in Example 2.1, it is probably that the milder diarrhea symptoms observed in the test group piglets (FIGS. 4 and 5) led the test pigs to consume more feed than the control pigs.

### Water Intake:

The increased food intake despite infection with F18 shown in FIG. 7 is also reflected in the water consumption for the test piglets.

FIGS. 9a and 9b show that the test group piglets consumed significantly more water than the control piglets. The is in line with the common general knowledge in the field that water intake and feed intake are positively correlated.

### Weight Gain:

FIG. 11 shows that accordingly, the test group piglets gained more weight than the control group piglets over a period of three weeks despite the occurrence of diarrhoea.

### Conclusion of Example 3.1

The results of Example 3.1 show that providing lactic acid, acetic acid and tannic acid in drinking water of weaning piglets does not have a negative effect on water and food intake. Conversely, test group piglets i.e., the piglets the piglets that were administered drinking water with lactic acid, acetic acid and tannic acid had a high water and food intake that piglets in the control group even when challenged with F18 ETEC bacteria.

Accordingly, test group piglets showed a higher weight gain in three weeks than the control group piglets, reflecting better general physiological wellbeing.

### Example 3.2: Water and Feed Intake

Lactic acid is known to have an unpleasant taste. The drinking water comprising lactic acid, acetic acid, tannic acid and polygalactose received by the test piglets should therefore be palatable so that the piglets consume a comparable amount of water to the control piglets.

### Purpose

The purpose of Example 3.2 is to analyse whether the drinking water comprising lactic acid, acetic acid, tannic acid and polygalactose is palatable to the piglets. The piglets should consume a volume of feed and water such that the beneficial effects of the drinking water additives are realized.

### Set Up

The content of the feed received by the test group piglets is shown in Table 7. The content of the drinking water received by the test group piglets is shown in Table 10. Drinking water for the test group piglets was administered via Dosatron (Dosatron©, France) at a setting of 3.7%. Control group piglets were administered only tap water.

TABLE 10
Composition of the drinking water administered by test group piglets.

| | Lactic acid | Acetic acid | Tannic acid | |
|---|---|---|---|---|
| | (80%) | (5%) | (100%) | Polygalactose |
| Stock | 3.6 L | 1.4 L | 124 g | 13.1 g |
| solution | (3.456 kg | (0.07 kg | | |
| | 100% LA) | 100% HAc) | | |
| Dilute to | 0.1728 g | 0.0175 kg | 0.0062 kg TA/L | 0.000655 kg |
| 20 L | LA/L | HAC/L | | PG/L |
| Fed to | 0.0064 kg | 0.00065 kg | 0.0002294 kg | 0.00002424 kg |
| test pigs | LA/L | HAc/L | TA/L | PG/L |

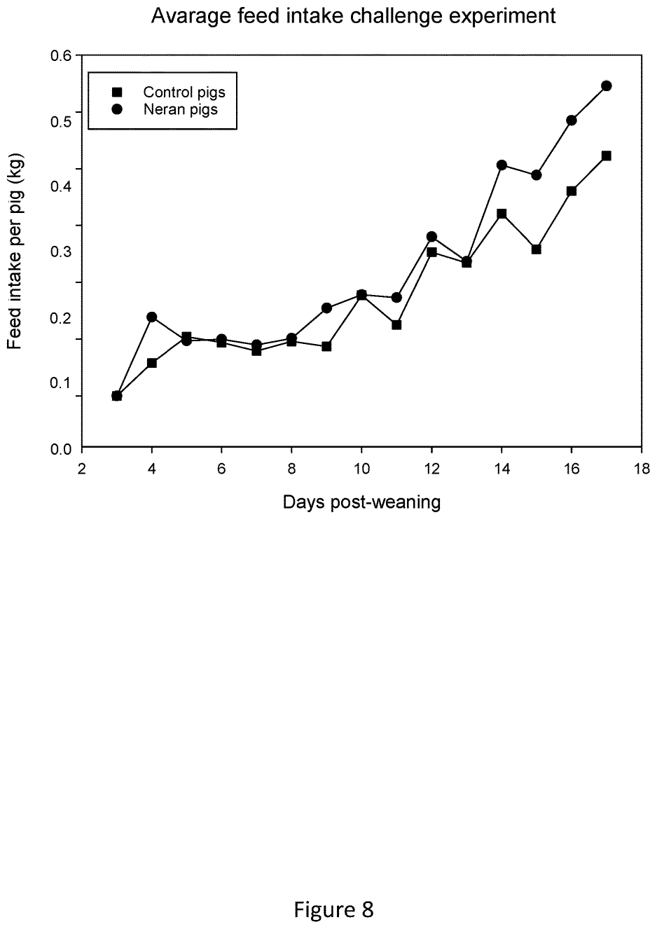

The feed intake of the control and test piglets was measured every day from day 0 to day 16, and the average feed intake calculated. Results are show in in FIG. 8.

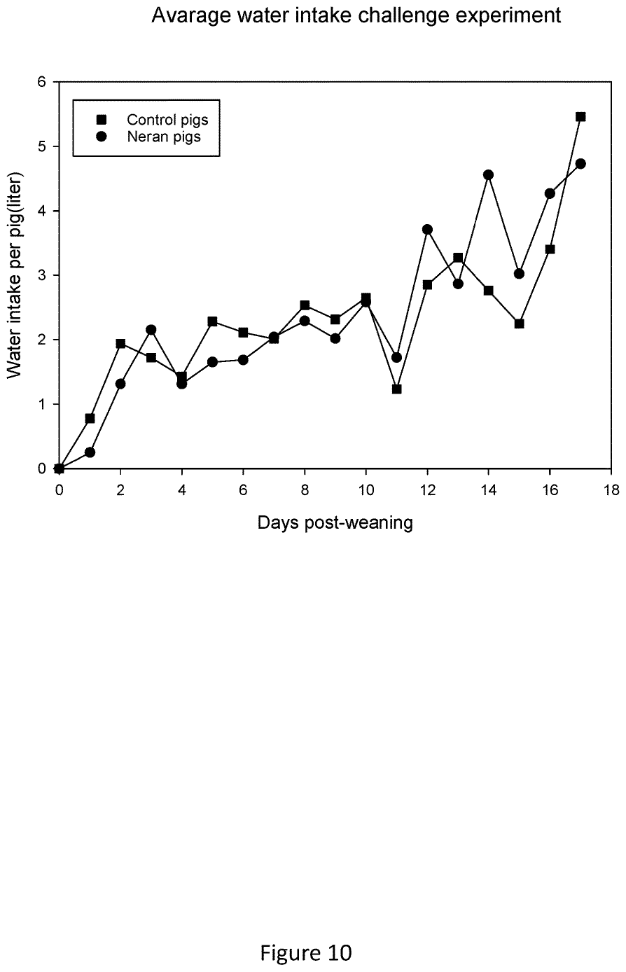

The average water intake of the test and control piglets was measured every day from Day 0 to Day 21. Results are shown in FIG. 10.

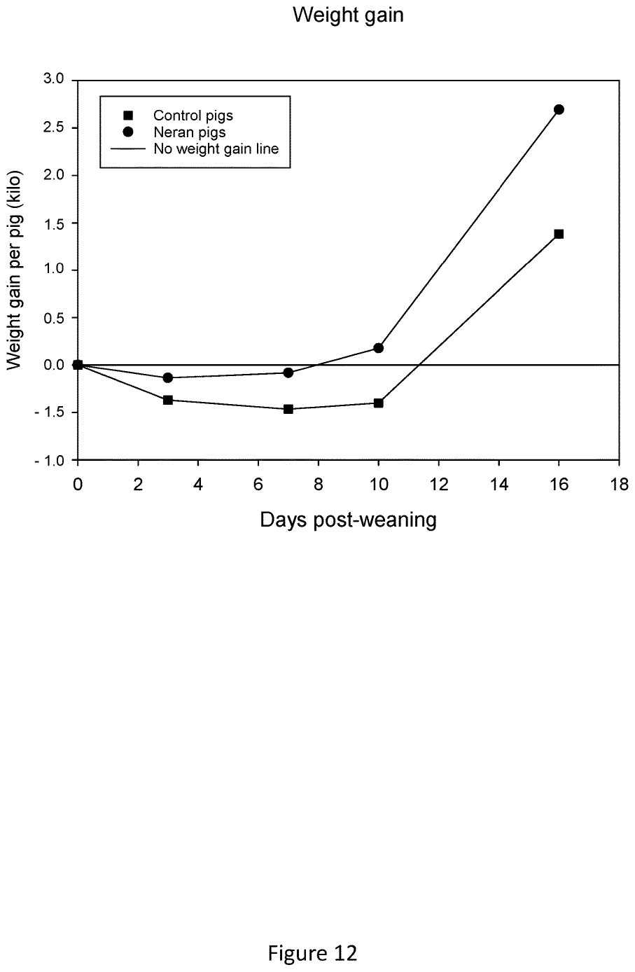

The test and control piglets were individually weighted on Day 0, Day 7, Day 15 and Day 21, and the average weight gain calculated. Results are shown in FIG. 12.

### Results

### Feed Intake:

The feed consumed per pig during the challenge experiment of Example 2.2 is shown in FIG. 8. FIG. 8 shows that the test group piglets consumed considerably more feed than the control group piglets. In accordance with the experiments in Example 2.2, it is probably that fewer diarrhea incident observed in the test group piglets (FIG. 6) led the test pigs to consume more feed than the control pigs.

### Water Intake:

The increased food intake despite infection with F18 shown in FIG. 8 is also reflected in the water consumption for the test piglets.

FIG. 10 shows that the test group piglets consumed more water than the control piglets. The is in line with the common general knowledge in the field that water intake and feed intake are positively correlated.

### Weight Gain:

FIG. 12 shows that accordingly, the test group piglets gained more weight than the control group piglets over a period of three weeks despite the occurrence of diarrhoea.

### Conclusion of Example 3.2

The results of Example 3.2 show that providing lactic acid, acetic acid, tannic acid and polygalactose in drinking water of weaning piglets does not have a negative effect on water and food intake. Conversely, test group piglets i.e., the piglets the piglets that were administered drinking water with lactic acid, acetic acid, tannic acid and polygalactose had a high water and food intake that piglets in the control group even when challenged with F18 ETEC bacteria.

Accordingly, test group piglets showed a higher weight gain in three weeks than the control group piglets, reflecting better general physiological wellbeing.

### Example 4.1: E. coli Agglutination Test

### Purpose

The purpose of the experiment was to test the effects of lactic acid, acetic acid and tannic acid (or combinations thereof) on E. coli cells producing F4 fimbriae. Without wishing to be bound by theory, it is thought that agglutination of E. coli cells prevents the cells from adhering to the intestinal walls and proliferating in the gastrointestinal tract. This in turn may prevent or inhibit infection with E. coli.

### Set Up

The effect of three different compositions on E. coli agglutination were tested: (i) lactic acid and acetic acid, (ii) tannic acid, (iii) lactic acid, acetic acid and tannic acid.

A recombinant E. coli strain constitutively expressing F4 fimbriae was washed three times with 0.9% sodium chloride, and cells corresponding to an optical density (OD450) of 1.5 were resuspended in 50 μL of 0.9% sodium chloride.

5 μL of the cell suspension was mixed with 5 μL of either (i) lactic acid and acetic acid, (ii) tannic acid or (iii) lactic acid, acetic acid and tannic acid on the surface of a glass plate.

The concentration of each of these components were identical to those delivered to the pigs in Example 3.1.

### Results

FIG. 13 shows the results of the E. coli agglutination test.

The first sample wherein the E. coli cells were mixed with a lactic acid and acetic acid shows very partial cell aggregation (small white dots in the droplet). The second sample wherein the E. coli cells were mixed with tannic acid does not show any significant aggregation of the cells. The third sample, wherein the E. coli cells were mixed with lactic acid, acetic acid and tannic acid shows full agglutination of the E. coli cells.

Only the composition comprising all three of the acids was able to fully agglutinate the F4 producing E. coli cells.

### Conclusion of Example 4.1

The results of the Example 4.1 show that a combination of lactic acid, acetic acid and tannic acid is effectively agglutinating E. coli cells.

It is therefore theorized that this combination would also be effective for preventing E. coli infection in piglets.

### Example 4.2: E. coli Agglutination Test

### Purpose

The purpose of the experiment was to test the effects of compositions comprising different binding agents and/or acid components on E. coli cells producing F4 fimbriae. Without wishing to be bound by theory, it is thought that agglutination of E. coli cells prevents the cells from adhering to the intestinal walls and proliferating in the gastrointestinal tract. This in turn may prevent or inhibit infection with E. coli.

### Set Up

The effect of different compositions on E. coli agglutination were tested against a control.

A recombinant E. coli strain constitutively expressing F4 fimbriae was washed three times with 0.9% sodium chloride, and cells corresponding to an optical density (OD450) of 1.5 were resuspended in 50 μL of 0.9% sodium chloride.

5 μL of the cell suspension was mixed with 5 μL of either a control containing 0.9% NaCl (Panel A) or a composition based on the composition to be tested (Panel B) on the surface of a glass plate.

The concentration of each of these components, when present, were identical to those delivered to the pigs in Example 3.2.

### Results

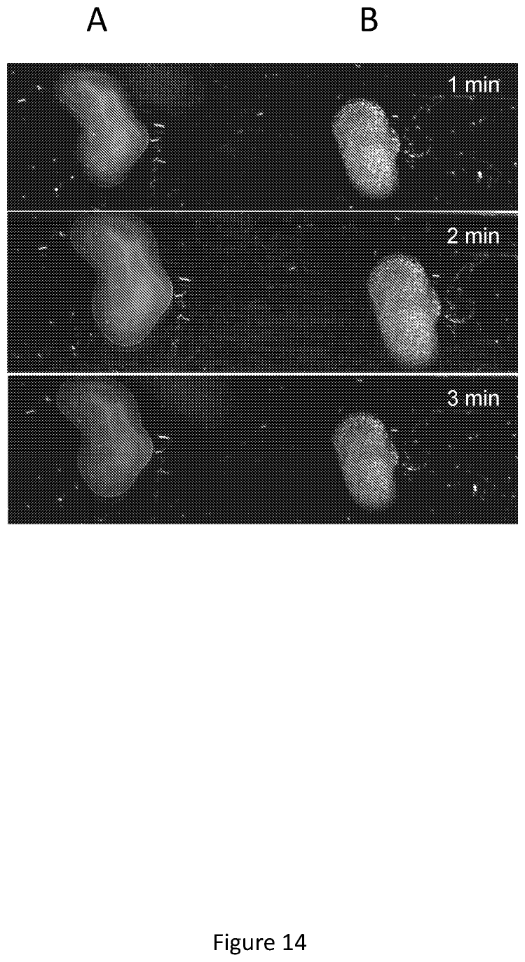

FIG. 14-18 shows the results of the E. coli agglutination test.

The aggregation experiments were conducted with various combinations of the components present in the liquid delivered to the pigs in Example 3.2. An overview of the experiments is seen below in Table 11:

TABLE 11
Overview of the agglutination experiments with
concentrations of each component identical to
those delivered to the pigs in Example 3.2.

| | Lactic | Acetic | Other | Tannic | | | Agglu- |
|---|---|---|---|---|---|---|---|
| FIG. | acid | acid | acids | acid | Polygalactose | pH | tination? |
| 14 | X | X | | X | X | 2.7 | Yes |
| 15 | X | X | | | | 2.7 | No |
| 16 | | | HCl | X | X | 2.7 | Yes |
| 17 | | | H₂SO₄ | x | X | 2.7 | Yes |
| 18 | X | X | | | X | 2.7 | Yes |

In each experiment, the control panel (Panel A) which was E. coli cells mixed with NaCl solution clearly did not cause cell aggregation. Furthermore, an acidic environment does not in itself cause cell aggregation. However, an acidic environment in combination with polygalactose and optionally acid causes the cells to aggregate (small white dots in the droplet).

The choice of acid is not important, but rather the pH of the composition appears relevant for causing aggregation.

### Conclusion of Example 4.2

The results of the Example 4.2 show that a combination of an acidic environment and polygalactose, optionally in combination with another acid is effective in agglutinating E. coli cells.

It is therefore theorized that this combination would also be effective for preventing E. coli infection in piglets.

1. A composition for an animal comprising and acid component and a binding agent, wherein;
the acid component comprises lactic acid and optionally a second acid and wherein;
the binding agent comprises a tannin component and a polygalactose component, and
wherein the tannin component is tannic acid or salt or ester thereof.

2.-9. (canceled)

10. The composition of claim 1, wherein the polygalactose component comprises 16 or more monosaccharide sub-units.

11. (canceled)

12. The composition of claim 1, wherein the polygalactose component is a carrageenan, preferably wherein the polygalactose component is kappa-carrageenan.

13. The composition of claim 1, wherein the second acid is acetic acid.

14. A composition for storage/transport comprising the composition of claim 1, wherein the composition for storage and/or transport comprises:
an acid component;
a binding agent; and
up to 20%, 30%, 40% or 50% (w/w) water.

15. A composition for administration comprising the composition of claim 1, wherein the composition for administration comprises;
an acid component;
a binding agent; and
up to 96.5%, 97%, 98%, 99% or 99.5% (w/w) water.

16. (canceled)

17. The composition for administration of claim 15, wherein the acid component(s) is in the range of 0.055% (w/w)-5.5% (w/w), such as 0.080 (w/w)-4 (w/w), e.g. 0.1 (w/w)-3 (w/w), such as 0.2 (w/w)-2 (w/w), e.g. 0.3 (w/w)-1 (w/w), such as 0.4 (w/w)-0.9 (w/w), e.g. 0.5 (w/w)-0.8 (w/w), such as 0.6 (w/w)-0.8 (w/w).

18. The composition for administration of claim 15, comprising a second acid, wherein the second acid is acetic acid, and is included in an amount in the range of 0.0014% (w/w) to 0.14% (w/w), such as 0.002% (w/w) to 0.1% (w/w), e.g. 0.004% (w/w) to 0.08% (w/w), such as 0.006% (w/w) to 0.07% (w/w), e.g. 0.008% (w/w) to 0.06% (w/w), such as 0.01% (w/w) to 0.03% (w/w), preferably 0.01% (w/w) to 0.015 wt %.

19. The composition for administration of claim 15, wherein the amount of binding agent is in the range 0.003% (w/w)-0.9% (w/w), for instance 0.005 (w/w)-0.8 (w/w), 0.006 (w/w)-0.5 (w/w), 0.008 (w/w)-0.2 (w/w), 0.01 (w/w)-0.1 (w/w), 0.02 (w/w)-0.1 (w/w), or 0.025 (w/w)-0.1 (w/w).

20. The composition for administration of claim 15, wherein the amount of tannin component is in the range 0.00259 (w/w)-0.9% (w/w), such as 0.003 (w/w)-0.8 (w/w), e.g. 0.005 (w/w)-0.5 (w/w), 0.008 (w/w)-0.7 (w/w), such as 0.01 (w/w)-0.6 (w/w), e.g. 0.01 (w/w)-0.5 (w/w), such as 0.015 (w/w)-0.4 (w/w), e.g. 0.015 (w/w)-0.3 (w/w), such as 0.02 (w/w)-0.2 (w/w), e.g. 0.025 (w/w)-0.1 (w/w).

21. The composition for administration of claim 15, wherein the amount of polygalactose component is in the range 0.000259% (w/w)-0.259% (w/w), such as 0.0003 (w/w)-0.2 (w/w), e.g. 0.0005 (w/w)-0.1 (w/w), 0.0008 (w/w)-0.1 (w/w), such as 0.001 (w/w)-0.1 (w/w), e.g. 0.0015 (w/w)-0.1 (w/w), such as 0.005 (w/w)-0.09 (w/w), e.g. 0.002 (w/w)-0.08 (w/w).

22. The composition for administration of claim 15, comprising 0.02-0.03% (w/w) binding agent, wherein the binding agent comprises a tannin component and a polygalactose component.

23. A feed, feed additive and/or premix wherein the feed comprises at least 0.05% w/w of the composition of claim 1.

24. A method of administering the composition of claim 1 comprising adding the composition to drinking water to provide a composition for administration.

25. The method of claim 24, wherein the animal consumes an effective amount of binding agent per kg body weight per day by consuming between 1.5 L and 5 L of the composition for administration per day, wherein the composition for administration is administered via drinking water.

26. The composition according to claim 1 for improving or maintaining the biophysical characteristics of animals compared to animals that do not receive the composition, wherein the biophysical characteristic is at least one characteristic selected from the group consisting of:
a) An increase in feed conversion ratio,
b) An increase in weight,
c) An increase in drinking water consumption,
d) An increase in the dry matter content of faeces,
e) A decrease in symptoms of PWD, and
f) An increase in feed consumption.

27. The composition according to claim 1, wherein at least 0.1 g binding agent is consumed per kg body weight per animal per day.

28. The composition according to claim 1 for increasing weight gain in weaning piglets relative to weaning piglets who did not consume the composition.

29. The composition according to claim 1 for:
preventing growth or decreasing the number, of E. coli in the gastrointestinal tract and/or
for increasing, maintaining or re-establishing dry matter content of faeces for piglets with post-weaning diarrhoea compared to post-weaning piglets with PWD that did not consume the composition.

30. The composition according to claim 1 for preventing and/or treating post-weaning diarrhoea in a piglet.
